(19) United States
(12) Patent Application Publication
(10) Pub. No.: US 2024/0232427 A1
(43) Pub. Date: Jul. 11, 2024

(54) CLOUD BASED VIEWING, TRANSFER AND STORAGE OF MEDICAL DATA

(71) Applicant: Datcard Systems, Inc., Irvine, CA (US)

(72) Inventors: Kenneth Wright, Chino Hills, CA (US); John C. Canessa, Apple Valley, CA (US); Gino Canessa, Eagan, MN (US)

(21) Appl. No.: 18/397,494

(22) Filed: Dec. 27, 2023

Related U.S. Application Data
(63) Continuation of application No. 17/875,212, filed on Jul. 27, 2022, now abandoned.
Application No. 17/875,212 is a continuation of application No. 16/582,776, filed on Sep. 25, 2019, now abandoned.
Application No. 16/582,776 is a continuation of application No. 15/372,314, filed on Dec. 7, 2016, now abandoned.
Application No. 15/372,314 is a continuation of application No. 14/084,472, filed on Nov. 19, 2013, now abandoned.
(60) Provisional application No. 61/729,306, filed on Nov. 21, 2012.

(51) Int. Cl. G06F 21/62; G06F 16/951; G06Q 10/10; G06Q 40/08; G16H 10/60; G16H 30/20; G16H 50/50

(52) U.S. Cl. CPC G06F 21/6245; G06F 16/951; G06Q 10/10; G06Q 40/08; G16H 30/20; G16H 50/50; G16H 10/60

(57) ABSTRACT

Features are disclosed for remote storage of medical information in a cloud service, and the systems and methods for sending, receiving, and accessing the data into and from the cloud server. A user may identify DICOM studies stored in a database at a medical data repository to be exported. The cloud service or an on-site unit at the medical data repository may search databases at the medical data repository for related medical information to identified DICOM studies. The identified DICOM studies and the related medical information may be burned into portable storage or combined into a virtual package to be uploaded to the cloud service. The cloud service may receive one or more recipients to grant access to the uploaded virtual package and may send an email to the one or more recipients indicating that virtual package may now be accessed.

### CROSS-REFERENCE TO RELATED APPLICATIONS

The present application is a continuation of U.S. patent application Ser. No. 17/875,212, filed Jul. 27, 2022, which is a continuation of U.S. patent application Ser. No. 16/582,776, filed Sep. 25, 2019, now abandoned, which is a continuation of U.S. patent application Ser. No. 15/372,314, filed Dec. 7, 2016, now abandoned, which is a continuation of U.S. patent application Ser. No. 14/084,472, filed Nov. 19, 2013, now abandoned, which claims priority to U.S. Provisional Patent Application No. 61/729,306, filed Nov. 21, 2012, the disclosures of which, including the appendices, are incorporated herein by reference in their entireties for all purposes.

### BACKGROUND

### Field

This disclosure relates to devices, systems, and secure methods of distributing private medical information among doctors, patients, imaging centers, medical centers, treatment centers, and hospitals, and more specifically, distribution of medical information via third party storage locations.

### Description of the Related Art

Hospitals and doctors' offices are the stewards of private patient medical information. Every time a patient visits a doctor, clinic or hospital, their private personal and medical information is recorded. The personal and medical information is stored in hospital databases, which can consist of picture archiving and communication systems (“PACS”), Radiology Information Systems (RIS), Hospital Information Systems (HIS), storage on imaging modalities, workstations, relational databases, fixed content storage systems, and computer files, among other storage methods.

Under certain likely scenarios, this personal and medical information must be accessible by medical personnel outside of a patient's typical doctor's office or hospital. It is not uncommon for a hospital to seek outside expert doctors to consult on interpreting lab results and medical images to improve chances of diagnosis. Another scenario requiring image transfer is when a patient is out of their normal living area, and another hospital requires use of medical images that are stored at the patient's local hospital. These outside hospitals and doctors require access to the medical information databases inside the doctor's offices and hospitals to make their diagnosis and perform their job. Similarly, a patient may seek an outside doctor's advice herself, either to see a specialist in a field, or to get a second opinion.

One option is to grant electronic access to the patient's information, but current hospital access systems have a number of issues. Hospitals are reluctant to grant access to their databases to outside doctors automatically, and often require that even internal doctors fill out paperwork, apply for access, and wait long periods before access is available. Further, many medical facilities require their doctors to remember and type into their computer complicated Uniform Resource Locator (URL) strings. Moreover, there is a lack of seamless access to the medical information held or controlled by a doctor, clinic, hospital, a third-party imaging center, or cloud-based medical data repository (collectively referred to as “MDRs”). MDRs are reluctant to provide seamless access to client entities for several reasons.

One reason MDRs are reluctant to provide seamless access to client entities is that MDRs contain private personal information. Unless an MDR restricts access to this information, the unauthorized release of a person's medical history and images could violate the patient's privacy and cause severe embarrassment. Thus, MDRs restrict access to a patient's medical records to small set of users, and carefully scrutinize any users applying for access to the information.

Another issue is that MDRs must comply with all current and future health information laws and regulations. One such federal regulation scheme is the Health Insurance Portability and Accountability Act (HIPAA) which regulates the use and disclosure of Protected Health Information. The regulations may require any access to equipment containing health information to be carefully controlled and monitored. Access to hardware and software must be limited to properly authorized individuals in some cases. HIPAA also may require authentication of any entity that communicates with an MDR, such as authentication through the use of corroborating password systems, two or three-way handshakes, telephone callback procedures, and token systems. HIPAA also seeks to ensure that the data within an MDR's systems have not been altered or accessed in an unauthorized manner. Any violation of HIPAA can result in an investigation by federal authorities and civil money penalties.

Thus, MDRs are reluctant to grant access to their electronic records. An outside doctor who requires access to patient medical information databases inside an MDR must often wait for months or years while an investigation occurs, and clearance procedures are performed. Consequently, many outside doctors avoid applying for direct access to hospital databases, and instead seek other methods of access to their patient's medical information.

Some doctors seek a physical delivery of electronic records to their offices for evaluation. These electronic records are often transported on a CD-ROM, DVD, or other portable storage media such as a USB key, memory card or stick, flash drive, thumb drive, optical disc, or portable disk drive. Either the patient requests the records from their MDR and supplies them for the doctor, or the doctor can acquire the portable media directly from the MDR. The doctor then can load the images from the portable media onto his local computer and use them for diagnosis.

There are numerous problems with accessing the medical information in this manner. Portable media has limited storage capacity, and the size of medical records and medical images have grown substantially. For example, image formats often are comprised of multiple 2D slice images to create a 3D image, growing an image files size. Further, if the images contain fourth dimension time information, the file sizes can grow rapidly. Thus, the larger hi-tech medical images may not be able to be transported by portable media, or would require additional portable media that consumes additional time, cost, and effort to create and transfer. Further, portable media is often accessed by the local reading computer at a slow rate compared to permanent media such as a hard drive. Thus, it may take a while for the media to load on the doctor's computer.

Additionally, many MDRs themselves need to store large amounts of data outside their hospital. First, storage space within a hospital's IT infrastructure may be limited. Thus, a hospital may need to use outside storage to store all medical images and reports for all their patients across their lifetime. Moreover, for safety or as required by law, a hospital may choose offsite storage of medical information to assist with emergency preparation. Storing images offsite preserves the information in case of a fire or other emergency that may affect local access to the data.

Thus, an offsite method of access that is responsive to the needs of security, health information laws and regulations, and ease of access is desired. These and other problems are addressed by the embodiments described below.

### BRIEF DESCRIPTION OF THE DRAWINGS

Throughout the drawings, reference numbers may be re-used to indicate correspondence between referenced elements. The drawings are provided to illustrate example embodiments described herein and are not intended to limit the scope of the disclosure.

FIG. 1a is a diagram of illustrative network(s) for various MDRs.

FIG. 1b is a diagram of illustrative network(s) for various MDRs that show an exemplary workflow for storing and sending medical information through the cloud.

FIG. 2a is a block diagram of an illustrative process to create users for a third party's medical information repository.

FIG. 2b is a block diagram of an illustrative process for modifying users of a third party's medical information repository.

FIG. 3a is a block diagram of an illustrative process for allowing users or organizations that are outside an MDR to view and/or download images and reports via a third party's medical information repository.

FIG. 3b is a block diagram of an illustrative process for sending medical information to recipients that are affiliated with an outside MDR.

FIG. 3c is a block diagram of an illustrative process for exporting data from an MDR to a CD/DVD or virtual package in a third party medical information repository.

FIG. 4 is a block diagram of an illustrative process for downloading data from a third party's medical information repository.

FIG. 5a is a diagram of illustrative network(s) showing processes for transferring data from a local MDR to a third party's medical information repository.

FIG. 5b is a diagram of illustrative network(s) showing processes for transferring data to a local MDR from a third party's medical information repository.

FIG. 6 is a block diagram illustrating a system and method for sending images through a third party's medical information repository that may require HIPAA release.

FIG. 7 is a block diagram illustrating a system and method for sending and receiving images through a third party's medical information repository that may require a HIPAA release and how the HIPAA release can be electronically gathered.

FIG. 8 is a block diagram of an illustrative process for downloading data from a third party's medical information repository and reconciling patient information between institutions.

FIG. 9 is a block diagram of an illustrative process for requesting and accessing data from a third party's medical information repository.

FIG. 10 is a block diagram of an illustrative process of gathering patient information to upload to a third party's medical information repository.

FIG. 11 is a block diagram of an illustrative process for allowing indefinite patient access to that patient's medical information stored in a third party's medical information repository.

FIG. 12a is a block diagram of an illustrative process for controlling access to patient information within a third party's medical information repository.

FIG. 12b illustrates sample records of one embodiment of a HIPAA status table(s).

FIG. 12c illustrates sample records of one embodiment of a patient reconciliation table(s).

FIG. 13 illustrates one embodiment of a sample user interface and functionality for accessing information sent and received by multiple organizations and roles affiliated with a user of the third party medical information repository.

FIG. 14 illustrates one embodiment of a sample user interface and functionality for accessing information sent and received by multiple organizations and roles affiliated with a user of the third party medical information repository.

FIG. 15 illustrates one embodiment of a sample user interface and functionality for accessing information sent and received by multiple organizations and roles affiliated with a user of the third party medical information repository.

FIG. 16 illustrates one embodiment of a sample user interface and functionality for accessing information sent and received by multiple organizations and roles affiliated with a user of the third party medical information repository.

FIG. 17 illustrates one embodiment of a system and method for adding one or more scanned images, reports, or other data to a DICOM study(ies) prior to storage of same.

FIG. 18 illustrates one embodiment of a system and method for adding one or more scanned images, reports, or other data to a DICOM study(ies) prior to storage of same.

FIG. 19 illustrates one embodiment of a system and method for adding one or more scanned images, reports, or other data to a DICOM study(ies) prior to storage of same.

FIG. 20 illustrates one embodiment of a system and method for adding one or more scanned images, reports, or other data to a DICOM study(ies) prior to storage of same.

FIG. 21 illustrates one embodiment for parsing DICOM headers that can be used for creating DICOM data for scanned data.

### DETAILED DESCRIPTION

Although several embodiments, examples and illustrations are disclosed below, it will be understood by those of ordinary skill in the art that the invention described herein extends beyond the specifically disclosed embodiments, examples and illustrations and includes other uses of the invention and obvious modifications and equivalents thereof. Embodiments of the invention are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the invention. In addition, embodiments of the invention can comprise several novel features and no single feature is solely responsible for its desirable attributes or is essential to practicing the inventions herein described.

In the following detailed description, references are made to the accompanying drawings that illustrate specific embodiments in which embodiments may be practiced. Electrical, mechanical, programmatic and structural changes may be made to the embodiments without departing from the spirit and scope of the disclosure.

Unless indicated otherwise, terms as used herein will be understood to imply their customary and ordinary meaning. Personal information is a broad term and is to be given its ordinary and customary meaning to a person of ordinary skill in the art (i.e., it is not to be limited to a special or customized meaning) and includes, without limitation, Social Security Number, address, phone number, email address, credit card numbers, bank accounts, and medical bills, and further would include identifying and person information relating to a particular person, including a HIPAA release.

Medical information is a broad term and is to be given its ordinary and customary meaning to a person of ordinary skill in the art (i.e., it is not to be limited to a special or customized meaning) and includes, without limitation, images, reports, exams, studies, lab results, test results, medical history, payment information, billing information, prescriptions and diagnoses, among other information.

Medical Data Repository (“MDR”) is a broad term and is to be given its ordinary and customary meaning to a person of ordinary skill in the art (i.e., it is not to be limited to a special or customized meaning) and includes, without limitation, any medical data repository, database, or storage media, which store medical information typically controlled by, for example, doctors, clinics, hospitals, lab centers, radiology centers, health insurance companies, etc.

Cloud, cloud service, cloud server, and cloud database are broad terms and are to be given their ordinary and customary meaning to a person of ordinary skill in the art (i.e., it is not to be limited to a special or customized meaning), and includes information storage and storage related services provided remotely by a third party to an MDR. A cloud service may include one or more cloud servers and cloud databases that allows for remote storage of medical information, hosted by a third party and stored outside of an MDR. A cloud server may include an HTTP/HTTPS server sending and receiving HTTP/HTTPS messages in order to provide web browsing user interfaces to client web browsers. A cloud server may be implemented in one or more actual servers as known in the art, and may send and receive medical information, user supplied information, or configuration data, among other data, that may be transferred to, read from, or stored in a cloud database. A cloud database may include a relational database such as an SQL database, or fixed content storage system, used to store medical information or any other configuration or administration information required to implement the cloud service. A cloud database may include one or more physical servers, databases, or storage devices that are necessary to implement the cloud service's storage requirements.

An on-site unit includes a unit related to the cloud service being provided, that is located within an MDR's facilities. The on-site unit may be owned and/or operated by the MDR, or by the cloud service. The on-site unit may include one or more of, a web server, a robotic-burner capable of burning DICOM CDs and DVDs, and/or local permanent electronic storage that may collect, store, and update medical information (such as DICOM studies and/or virtual packages) and medical information metadata in flat file systems, relational databases, or a fixed content storage. The on-site unit may also comprise separate devices that provide the same functionality, for example, a separate web server, an external robotic burner, and/or local storage implemented in a separate networked attached storage (NAS) device. The on-site unit may also be used to implement user interface features for the cloud service. For example, it may use its web server to send and receive user interface commands and display or transfer data to a client device in lieu of the cloud service doing so. In some embodiments, the on-site unit may also transfer some of this information, for example uploaded images (or other medical data) and/or user interface commands to cloud service, and thereby act as a proxy or bridge between the client device and the cloud service.

A user is a broad term and is to be given its ordinary and customary meaning to a person of ordinary skill in the art (i.e., it is not to be limited to a special or customized meaning) and includes both an organizational user, and a recipient. A user may refer to a user's account on the cloud service or an on-site unit. An organizational user is a user with an account on the cloud service and/or on-site unit, where the user and his account is affiliated with an MDR that has the capability of storing its medical information in the cloud service. A recipient is a type of user that may be sent images via the cloud service by an organizational user. A recipient may be an organizational user of another organization or may not be affiliated with an organization that stores its images in the cloud.

Image or images is a broad term and is to be given its ordinary and customary meaning to a person of ordinary skill in the art (i.e., it is not to be limited to a special or customized meaning) and includes DICOM studies that contain one or more series of images or reports.

A virtual package is a collection of DICOM studies, or links to or identifiers for DICOM studies, as explained herein. It may include only a single DICOM study, or multiple studies (and/or pointers thereto). Unless otherwise specified, any reference within this disclosure of a virtual package or a DICOM study (or study), can refer to, in various embodiments, both virtual packages and DICOM studies.

Details regarding several illustrative preferred embodiments for implementing the system and method described herein are described below with reference to the figures. At times, features of certain embodiments are described below in accordance with that which will be understood or appreciated by a person of ordinary skill in the art to which the system and method described herein pertain. For conciseness and readability, such a “person of ordinary skill in the art” is often referred to as a “skilled artisan.”

Various embodiments overcome one or more issues with the prior art. Other embodiments may overcome different issues with the prior art. For example, some of the embodiments herein provide for external MDR access to cloud stored information controlled by an MDR. Other embodiments provide for secure or documented/audited access to medical information.

It will be apparent to a skilled artisan, in light of this disclosure, that the devices, systems, and methods described herein can advantageously be implemented using computer software, hardware, firmware, or any combination of software, hardware, and firmware. In one embodiment, the system is implemented as a number of software modules that comprise computer executable code for performing the functions described herein. In one embodiment, the computer executable code is executed on one or more general purpose computers. However, a skilled artisan will appreciate, in light of this disclosure, that any module that can be implemented using software to be executed on a general purpose computer can also be implemented using a different combination of hardware, software, or firmware. For example, such a module can be implemented completely in hardware using a combination of integrated circuits. Alternatively or additionally, such a module can be implemented completely or partially using specialized computers designed to perform the particular functions described herein rather than by general purpose computers.

The foregoing and other variations understood by a skilled artisan can be made to the embodiments described herein without departing from the spirit of that which is disclosed herein. With the understanding therefore, that the described embodiments are illustrative and that the scope is not limited to the described embodiments, certain embodiments are described below with reference to the drawings.

### Introduction

The present disclosure is directed to providing access to data generated within and/or stored by a local MDR. Specifically, the disclosure relates to a system and method for storing medical information, including DICOM images and medical reports. The disclosure allows for the transfer of medical information from one hospital to another hospital (or from any MDR to another MDR) via storing the medical information in a cloud.

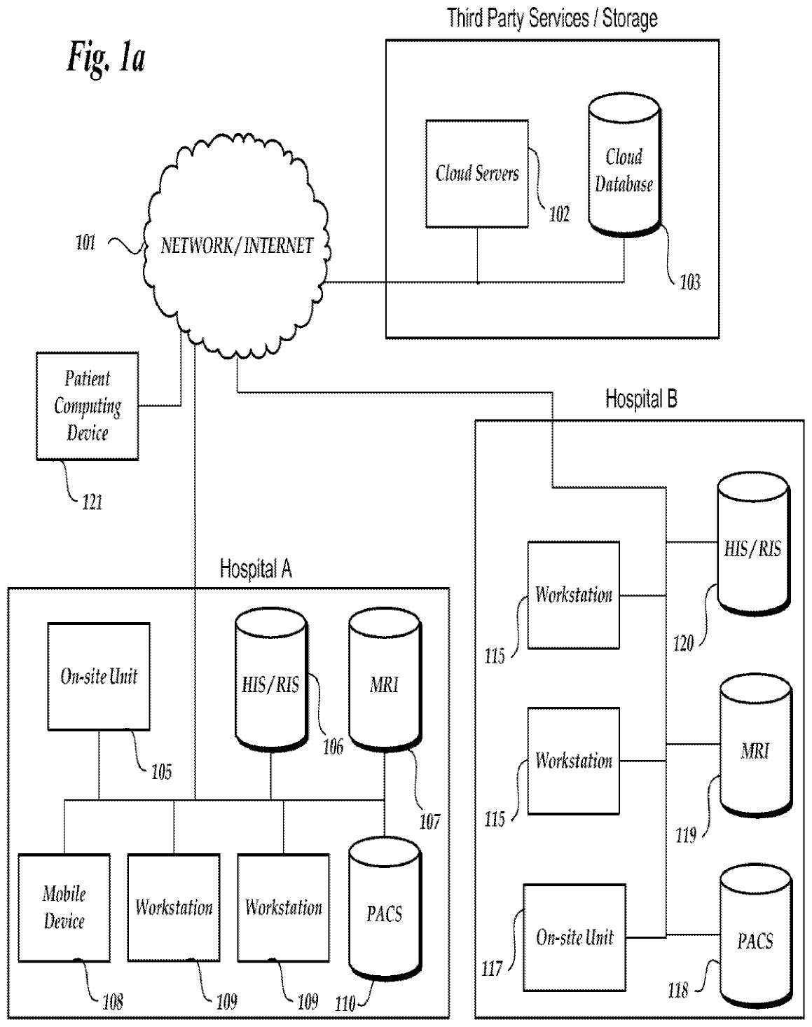

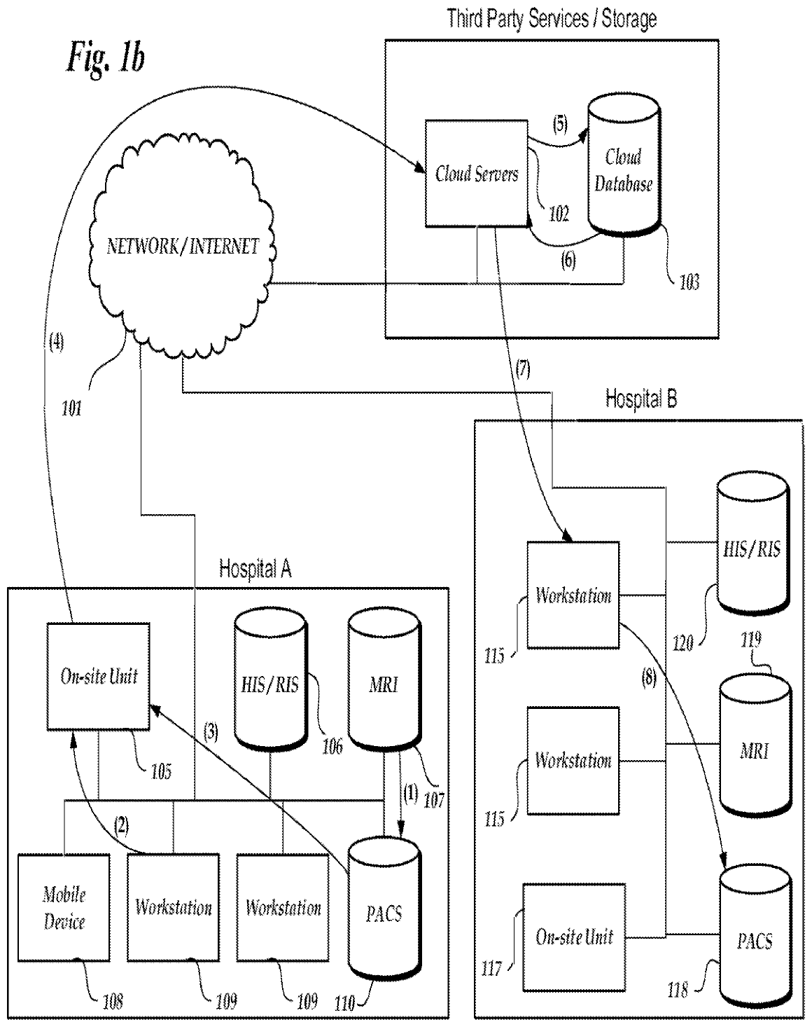

Referring to FIG. 1a as an overview by way of example, in some embodiments, a modality, such as MRI 107, at example hospital A may be used by a radiological technician. The patient is suffering from back pain and requires an MRI of his lower back. The radiological technician uses the MRI to generate DICOM images of the patient and may write a report about the procedure. The images and the report, combined into a DICOM study by the MRI, may be transferred to PACS 110 for permanent storage (1). A doctor may review the images and make a diagnosis. However, often, as here, the doctor may need to send the image off to another doctor or institution for additional analysis. The doctor may instruct hospital staff to send the images to this other entity.

The hospital staff, using a normal workstation 109 (or mobile device), may login to the on-site unit via a web browser (2) (or mobile app), which in some embodiments, may comprise a networked robotic CD/DVD burner and local storage. On the on-site unit 105, the hospital staff user enters search criteria to search the PACS for the patient's recent MRI's. A list of results is returned to the workstation, and the hospital staff user selects the corresponding MRI generated study on the PACS to send. The user also selects that he would like to send other related data about the patient and decides to send the image via the cloud service that the hospital subscribes to (rather than burning a CD/DVD). The on-site unit performs a search of configured databases, such as the PACS or HIS/RIS to gather related data about the patient, for example, x-rays of the patient's lower back from two months previous. All of these studies are transferred to the on-site unit, for example, via DICOM query/retrieve (3). The user inserts or selects the email address of the recipient to receive the studies. In this scenario, it would be the email of the outside doctor or the outside doctor's institution.

The study(ies) can be assembled into a virtual package. The virtual package can then be sent, via DICOM, HTTP/S, or another protocol, to the cloud server (4), along with any other data required to send store and send the virtual package, such as the recipient(s). The cloud server parses the virtual package for metadata, and stores the metadata, virtual package identifiers, recipient(s), studies, and related information into the cloud database (5). The cloud server may also send an email to the recipient'(s) email address. The email may contain a link to the images, and/or a link to register as a recipient user of the cloud service if the email address is not already affiliated with a recipient.

The recipient, in hospital B, assuming he has already registered with the cloud service, may now login, access their inbox of various studies, and download, via DICOM or HTTP/S, all the studies in the virtual package to their workstation 115 (7). Once downloaded, the studies can be sent to hospital B's PACS for storage using DICOM. Now the images and reports are available for use by a doctor at hospital B for diagnosis. At either of these steps, the cloud service and/or workstation may search hospital B for the patient's patient ID and other patient information specific to hospital B. This hospital specific data can then be used to update and reconcile any DICOM header tags that were specific to hospital A, via either a manual or automatic process. Alternatively, a doctor at workstation 115 could have viewed the sent studies online using their web browser via direct interaction with the cloud servers. In this scenario, there is no need for hospital specific data translation.

### Operational Environment

FIG. 1 is illustrative of a sample computer network that includes multiple enterprise networks that provide various information technology services.

Turning now to FIG. 1, example Hospital A's enterprise network contains a variety of medical facility specific networked devices. This includes one or more HIS (Hospital Information System) or MS (Radiology Information Systems) 106 like systems that store data about patients. HIS systems are comprehensive, integrated information systems designed to manage the medical, administrative, financial and legal aspects of a hospital and its service processing. For example, a HIS system may store patient medical records, including medical images (such as DICOM studies), diagnoses, performed procedures, results, reports (such as DICOM structured reports) etc. A HIS system may also store billing information, health insurance information, or audit information (for example, an access history for a specific patients' records). A MS is a computerized system and database typically used by radiology departments to store, manipulate, and distribute patient radiological data, imagery, and reports. It can include image and report tracking capabilities, patient scheduling and workflow capabilities. HIS/MS are often able to communicate with a hospital's IP (or other OSI layer 3) network and networked devices, and transfer medical information thru the network to/from other hospital information systems, such as a mobile device, workstations 109, PACS 110, and modalities. This transfer can occur using DICOM query/retrieve or other protocol such as HL7, FTP, etc.

Hospital A's network can also include one or more modalities, such as an MRI 107. A modality is an instrument that can acquire images of the body, such as radiography, ultrasound, and magnetic resonance imaging (MRI), among others. These instruments can obtain a patient's images and convert such native images into DICOM series and studies. The modality can also create, automatically or with input from medical personnel, reports, including DICOM structured reports that can be included within a DICOM series. Modalities are often able to communicate with a hospital's IP (or other OSI layer 3) network, and transfer medical information thru the network to/from other hospital information systems, such as a mobile device, workstations 109, PACS 110, HIS or RIS 106. This transfer can occur using DICOM query/retrieve or other protocol such as HL7, FTP, etc.

Hospital A's network can also include one or more PACS (Picture Archiving and Communication Systems), such as a PACS 110. A PACS provides economical storage of, and convenient access to, images from multiple modalities. Usually, DICOM images and structured reports are transferred via the network to a PACS device. PACS and related PACS workstations (not shown, although a normal workstation 109 may work with a PACS) may be used to view medical images, read structured reports, transfer medical information and diagnose patients. PACS are often able to communicate with a hospital's IP (or other OSI layer 3) network, and transfer medical information thru the network to/from other hospital information systems, such as a mobile device 108, workstations 109, HIS or RIS 106. This transfer can occur using DICOM query/retrieve or other protocol such as HL7, FTP, etc.

Hospital A's network can also include an on-site unit 105. Such a device may comprise, for example, a PACSCube system as developed by DatCard Systems, Inc. Such a system may comprise the production station described in U.S. Pat. No. 7,302,164, the disclosure of which is fully incorporated by reference herein, and is also attached hereto and made a part of this application. Such a system may include a variety of components to assist in exporting images from the hospital. For example, the on-site unit 105 may include robotic CD/DVD burning capabilities, and local storage to assist in burning medical information onto DVDs. This storage may be used to temporarily (or permanently) store DICOM images and/or reports generated by a HIS/RIS or modality. The local storage may include a local processor and database (SQL, fixed-content storage, etc.) to catalog the DICOM studies comprising medical images and reports, store extracted meta information (such as patient id, etc.) about the DICOM studies received, and create jobs to be burned by the CD/DVD burner. The local storage (which also may be independent from a robotic CD/DVD burner) may also be a part of a broader network for fixed content storage, as described herein. In addition, the on-site unit may include a web (or other networkable) interface, such as a web server, that can be used to login to the on-site unit, configure the on-site unit and its components, search for images and reports on the hospital network based on patient or procedure information (e.g. patient id, name, type of image etc.), send or receive images from a third party using the embodiments herein, track and audit access to patient medical information, create CD/DVD burning jobs, etc. Any of these components and features of the robotic burner may be implemented together in a single device, or separately among several devices with or without the robotic burner.

Workstations 109, and mobile devices 108 may also be attached to a hospital network, and be configured to view and access medical information on a HIS or MS 106, modality such as an MRI 107, a PACS system, or the on-site unit. This can be accomplished by medical personnel using a web browser or other propriety software (such as a PACS software package) on the workstation to access, view, retrieve, and store DICOM images, view update reports, etc. Workstation/mobile device software or a web browser can also be used to communicate with the on-site unit 105 to initiate and perform the tasks described above that may be carried out by device(s) 105.

Example Hospital B's network may include devices with similar functionalities as example Hospital A, including workstations 115, mobile devices (not shown), on-site units and other functionalities 117, PACS 118, modalities 119, and HIS and MS 103 systems.

An example third party may host cloud services for the storage of medical data, including images (DICOM or other formats), reports (DICOM structured or other formats), audit information, user information, authorization configuration for users and data, user authentication information, etc. To offer cloud services, these parties may use cloud servers 102 that often comprise a web server, or multiple web servers, that can be accessed through a web browser or other custom client application, or via web service, DICOM query/retrieve, or other method of invoking remote transfer and medical data related processes. The cloud servers 102 may store and retrieve medical information into/from a cloud database.

In one embodiment, the cloud database may comprise a fixed content storage system. A fixed content storage system is one that stores data which does not change over time. Such a system is ideal for storing medical imaging because medical images, once created, are typically not updated. Instead, if new images are taken of a patient, those images will be stored separately in a database rather than update the old images. A fixed content storage (FCS) system (sometimes referred to as a Content Addressable Storage system (CAS), though the system need not be strictly “content addressable”) is a file system that will accept data to be stored, and return a “ticket” or address that can be used to retrieve the file from the file system. An FCS system may compromise multiple file servers located locally, or remotely. For example, as a part of a single FCS system, there may exist FCS storage servers that are located at the third party location, and others that are located in Hospital A (for example, as a part of the on-site unit or separate). This would allow for a copy of medical images to be stored locally at the hospital, or in the third party cloud server, or both. Moreover, FCS systems may include their own requests and searching protocols, and thus a request for a file with a certain “ticket” or address may be forwarded from the servers at the third party location to the server at Hospital A, if the cloud did not contain the file associated with that ticket. For more information on FCS (CAS) systems, please refer to U.S. patent application Ser. No. 13/092,229, published as 2012/0005252, the disclosure of which is fully incorporated by reference.

In addition to the FCS storage system, the cloud database 103 may also comprise more traditional databases, such as an SQL database or other relational database, that can be used to store meta information about the files stored within the FCS system. For example, when a DICOM study is received, it may contain multiple series of images and reports related to on ore more patients. DICOM tags, and other information provided by the sender, may contain meta information, including a patient name, modality used, type of modality used to create the images, the date when images were created, a patient id, diagnosis, or any information or identifier about the images or patient (or the medical staff involved). This information can be stored in association with the location of the actual file or ticket/address so that the file can be retrieved based on the metadata stored.

In addition to medical information stored within cloud database 103, other information can be stored, such as information about subscriber organizations to the third party's service, associated users, permissions, recipients of images, HIPAA releases, etc. More information about this type of information and its use is described herein.

A third party cloud medical information service may be connected by a wide area network, for example the Internet 101, to enable data transfers between institutions, such as between any combination of the third party cloud service, hospital A and hospital B. Such a network can consist of a series of networks where packets can be forwarded between networks by routers and can route packets to their eventual destination.

### Organizational Users and Administrators

In some embodiments, one of the primary interfaces with the third party's medical information cloud service is a web interface. A web browser may be launched, for example, on a workstation within Hospital A. The user of the workstation may access an internet address or Uniform Resource Locator (URL) associated with the cloud service. Each user may correspond to a user entry within the cloud database 103. Each user entry may have a variety of attributes, roles and/or rights associated with the user, as described below. Alternatively, a custom application such as a mobile application for an iPhone or Android platform, as opposed to a browser, may be used to implement the same functionality.

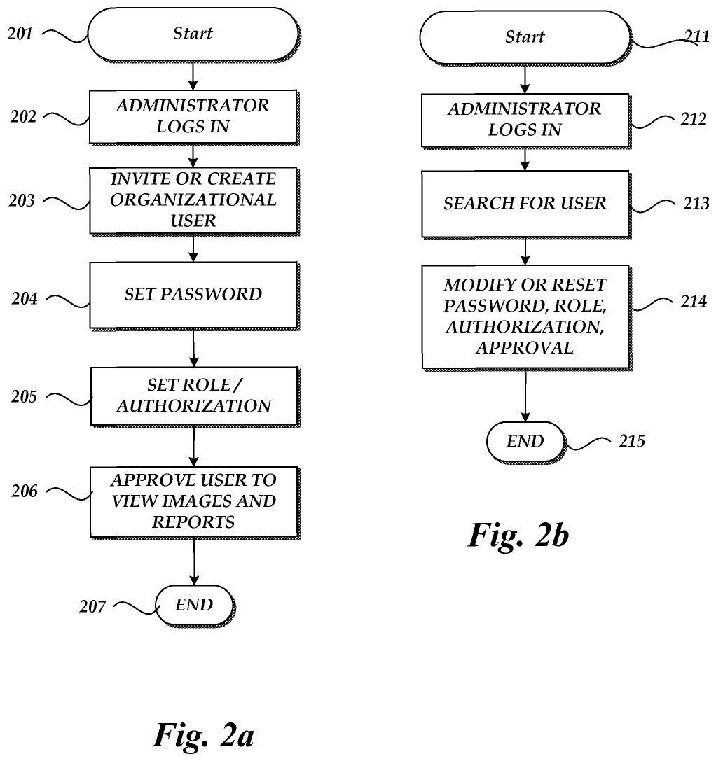

An organization, for example hospital A, may be initialized with a starting account that has administrator access (or role) for their organization (e.g. hospital A). Referring to FIG. 2a, the administrator may access a URL, and be prompted to login 202. Such a login may accept a username, such as an email address, and password for authentication, or involve any other authentication method including a certificate, knowledge based questions/answer, smart card, etc. The username and password (or other credential) is sent via HTTP (or HTTPS) to the cloud server and verified against usernames and passwords (or other credential) within the cloud database. If a match exists, and the user has access to the application, then access is allowed.

Once logged in, the user is presented with a variety of menu options transferred via HTTP (or HTTPS) to the user's web browser. One of the options is to invite or create another user within the cloud's service 203. This option may be presented along with other site administration options. Under this option, the administrator can create a user that is affiliated with the same organization (here hospital A) that the administrator is affiliated with. In this manner, the administrator may manage the users associated with his or her organization. The affiliations for each user may be stored within the cloud database 103.

To create the user, the administrator can enter the new user's email as his username (or whatever string is being used as the username) and an initial password (and/or have the system create one randomly) 204. In some embodiments, the administrator can specify that upon first login the new user must reset his/her password. Other data may be collected about the user as well, such as his or her full or partial name, position at the organization, contact information (telephone, address, email, etc.).

The administrator may also set the new organizational user's role within the cloud software. These roles may be configurable by the administrator and can be used to restrict access to various functionality of the cloud software. For example, one defined role may be “administrator” (the names for these roles are arbitrary and could change to other names in other embodiments). A user with this role may depending on configuration, be allowed to access the full functionality of the cloud website for that specific organization, just like the administrator described above. Another possible role is the viewer role. This user may not be able to access any functionality in the cloud's service besides being able to login and view images owned by or sent to the organization. Another possible role is the validator role. This user may view images just like the viewer but may also validate other non-organization recipients (explained later) to view images sent by the organization. A user may have more than one role.

As described above, the administrator (or validator, or any user with appropriate rights) may approve a user to view images and reports. By default, a user may not view any images or reports. This may be advantageous for security reasons, because, for example, access to view images may be restricted to doctors or other important medical personnel. A user may be given access to the system to view an audit trail, or access logs, without being able to actually view any images in order to reduce the risk of any one user misusing images available to him or her.

As a skilled artisan would recognize, such attributes about users (name(s), passwords, other authentication credentials (knowledge based questions/answers, contact info, roles (validator, etc.) and rights (view images/reports)) may be kept in the cloud database 103 in one or more SQL tables.

At any point in the organizational user creation process, an email may be sent to the new user's email address. Such an email may include a link that calls a URL associated with the cloud servers. When the URL is clicked, the URL is accessed and indicates to the cloud server that the user's email address actually exists and is valid. The URL accessed may have a special random number or key included within the URL to carry out the registration. Through this feature, it would be difficult for another person or process to guess the URL parameter without receiving the email.

Referring to FIG. 2b, an administrator may login 212 and modify a user's information at any time. For example, after logging in 212, an administrator may search for a specific user 213 associated with the administrator's organization by any of the user attributes, such as name, email, role, etc. After selecting the desired user, the administrator may modify various attributes of the user discussed above. Such modifications will update the cloud database about the user, and may reflect changes to various roles and rights of a user when he or she logs in.

### Recipient Users

Other users not associated with an organization may receive images sent by users at the organization. For example, a hospital may need to send images and/or reports to an outside doctor based on referral, or to outsource diagnosis, or for a variety of medical reasons. The outside doctor is not affiliated with the hospital, so does not have rights that may be associated with organizational users and cannot.

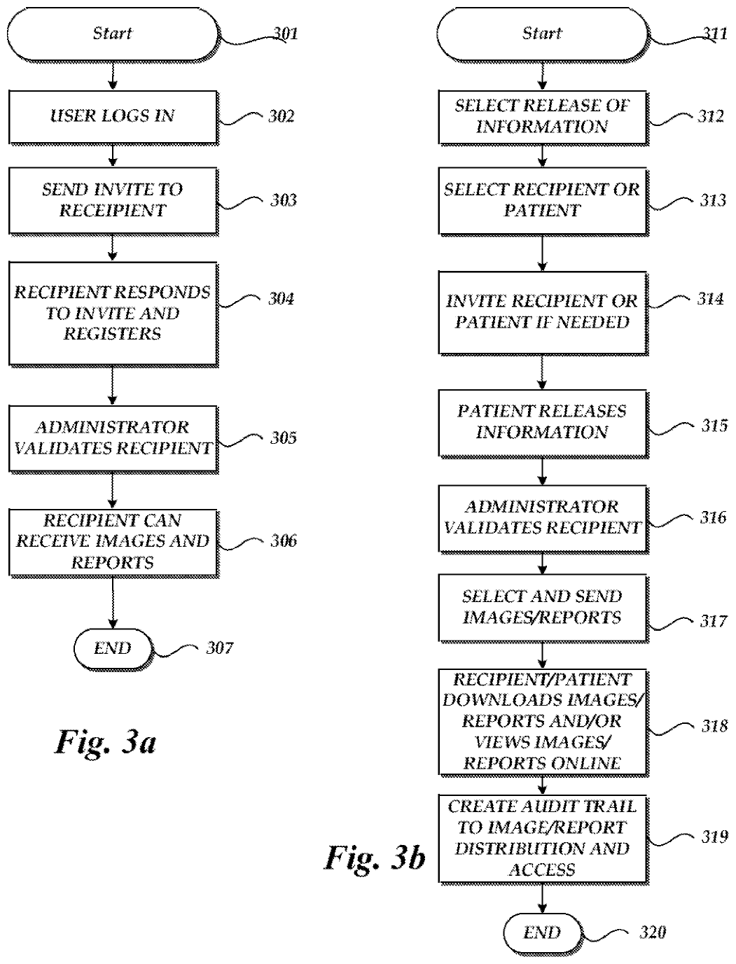

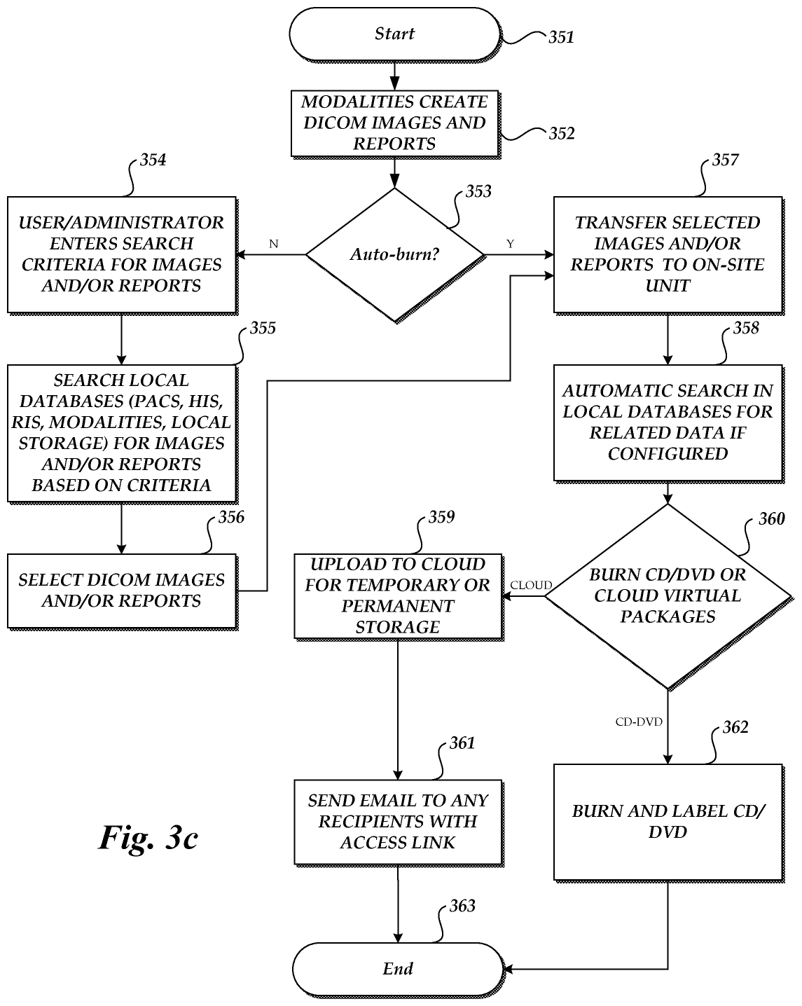

In some embodiments an organizational user may add a recipient type user not affiliated with the organization in order to send that recipient user DICOM images and reports for viewing. Referring to FIG. 3a, a user logs into the cloud based system 302. The user may then add a recipient user directly, by filling in all the attributes about the recipient user as discussed above for a normal organizational user. In some embodiments, a recipient user may also instruct the cloud system to send an invite to a recipient 303 to allow the recipient to enter collected attributes about the recipient user.

For example, a user logs in to the cloud system 302. After navigating the menus, the user selects a menu option to invite a recipient and is presented with a user interface to enter the recipient user's email address, among other attributes. The user may enter an email address which is recorded within the cloud database (and may enter other attributes, such as name(s). initial passwords, contact information (country, phone number, address, city, state/province/region, postal or zip code, language preference, etc. affiliated national provider number, etc.). The cloud server may then send to the recorded email address an email containing a URL link to the cloud server or a related server 304. If the recipient of the email accesses the URL associated with the link, the cloud server presents the recipient with an interface where they may enter attributes associated with a new recipient user account, such as name(s), passwords etc., if not entered by the user adding the recipient (or in addition to). By entering these attributes, the recipient user may register his account with his data recorded within the cloud database 304.

In some embodiments, the recipient may now receive DICOM images and reports sent by the organization that invited the recipient to join the system (sending and receiving described elsewhere). Alternatively, or in combination, in some embodiments, a recipient may be required to be validated 305 before a recipient can receive and/or view any sent images and/or reports. This validation or a recipient user may be configured to be performed only by an administrator after reviewing the recipients email address and/or other recipient attributes that are recorded within the cloud database. Furthermore, a recipient user may be validated on a per organization basis. For example, the same recipient user may be associated with multiple organizations and may be “validated” to view images from one organization, and not “validated” to view images and reports from another organization. In this way, the rights of a recipient user can be configurable for each individual organization as set by each organization's administrator(s).

Organization users or recipients may reset their password if forgotten (or for any other reason) at any time, by either typing in their old password, providing a knowledge based answer to a knowledge based question stored in the cloud database, or following an emailed link delivered via email to the account's email address. For the knowledge based method, the user is presented with their own knowledge based question that they selected or enter upon registration, and if they respond with the appropriate answer, then they are considered to be the authentic user or recipient and may reset their password. Likewise, for the email based method (which may be used in combination with the knowledge based method), an email with a verification link that contains a unique or rare indicator that, when accessed, instructs the cloud server that the user is authentic, and the user/recipient may reset their password.

### General Workflow to Send Images

In a high level view, there are two ways images may be sent to a recipient: 1) sending images already stored in the cloud's database 103, or 2) sending images that have not yet been transferred to the cloud's database. FIG. 3b focuses, by way of example, on sending images that are already uploaded to the cloud. However, nothing in this figure should be construed as applying only to images sent to a receiver that have already been stored in the cloud. The concepts described by this figure apply equally to sending images that are not yet stored in the cloud.

Turning to FIG. 3b (which, as discussed previously, may be done in other orders other than the example order depicted), in some embodiments, prior to sending DICOM images and/or reports to another organization via the cloud, a user must log in to the cloud's web interface. This user, in order to send images/reports, may be required to be validated, or required to have a certain role within the organization that controls the images/reports (e.g. only administrators and sender roles can send, or only administrators and viewers can send, etc.).

Assuming all required rights are satisfied, some embodiments may also require the sender to select whether a patient release is required, and if so, which patient release is required. Because medial images and reports about a patient are sensitive information, this information is controlled by the patient under the Health Insurance Portability and Accountability Act. In order to send medical information to a third party, an organization may be required to obtain a release or waiver of rights by the patient. Such a release/waiver may be a manual process where a patient signs and returns a release/waiver that is stored at the cloud and/or at the organization/medical facility. Such a physical copy may also be scanned and stored electronically in the cloud or organization/medical facility. For example, in block 312, if required, a user may select which specific release (or version of a release) is required to be signed before information can be sent regarding a specific patient.

In block 313, the user may select a recipient or patient to send images to. This recipient may already be in the system and validated, as per the functionality depicted in FIG. 3a, or the recipient may be invited to participate 314. If so, similar steps as described above for FIG. 3a may be employed to have the recipient or patient become registered with the cloud system. If the patient is already in the system, the user may enter search criteria (for example, a partial or full name or email address). The cloud system may search all recipients for matches, and return a list of matching recipients (or, potentially organizational users) which may receive the

In block 315, the patient releases may be gathered. This may be done via a manual process. For example, while present at a clinic or hospital, the staff may present a waiver/release to the patient to sign. Once signed, if required, the clinic or hospital may send an indication to the cloud that the waiver was signed, which may be recorded in the database in association with a specific patient or specific DICOM images and/or reports. In some embodiments, the waiver/release can be signed, scanned, and uploaded to the cloud system for future reference. More information on using electronic means to gather HIPAA releases are detailed elsewhere herein.

In block 316, an administrator may validate a recipient, if such a recipient wasn't validated previously, or if that patient was recently invited to the system (and subsequently registered) in block 314.

In block 317, the user sending images to the recipient may search for specific images and reports based on various criteria, including patient ids, names, emails, dates of procedure, types of procedure, email addresses, etc. Based on search results provided by the cloud server, the user may select one or more studies (also known as virtual packages) to send.

In block 318, the recipient may receive an email indicating that images have been sent to his cloud account and may be provided a URL link to a cloud server to view or download the images online. By clicking on the link, or alternatively browsing to the cloud site, the recipient may download and/or view the images online, as is disclosed below.

In block 319, the cloud server may create an audit trail, which may include a text log and/or database entries that indicate who sent and received the images/reports, when the images/reports were sent and received, what images/reports were sent and received, any patient information related to the images/reports (patient id, name, etc.), a tracking number assigned to one or more groups of images/reports sent, each time the images were accessed and by whom, etc. Such logs or database entries may be searched or downloaded via the web interface to, among other uses, determine who accessed what images/reports and when.

### Storing Images to the Cloud

FIG. 3c describes an illustrative workflow to transfer images from one medical facility (MDR, etc.) to another. Often, modalities located in hospitals, clinics, radiology labs, etc., are used to generate images and/or reports of patients 352. These images can be locally stored in the modality's storage in DICOM format.

DICOM can store multiple studies for a single patient, where each study can contain multiple series of images. For example, a patient enters a hospital and is given a patient ID. After review by a clinician, the patient is sent to radiology to have a variety of images taken, for example a CAT scan and an MRI. The order for radiology may be performed by a HIS or RIS and support a modality worklist that the modalities can refer to in order to gather information about the patient, including patient ID, and a DICOM accession number. The accession number will apply to both the CAT scan and MRI because they are a part of the same order (i.e. accession number can be used to indicate a single order for a patient, or even a single visit by a patient to radiology). The CAT scan, and the MRI will both produce a DICOM study, which can consist of one or more series of images collected by the modalities.

At this point, a modality, such as the MRI modality or CAT scan modality referred to above, has stored in its local storage (or related storage, such as a PACS or RIS) the study it has created 352. At this point, the on-site unit may determine whether auto-burn is configured for the modality (or PACS or RIS) storing the images that were just created.

In some embodiments, an auto-burn feature allows a patient's images recently created by a modality to be automatically exported to a CD or to the cloud. For example, to determine whether images have been recently created for a patient, an on-site unit 105 may monitor images stored by modalities 107, PACS 110, a HIS or RIS 106, or workstations. It can perform such monitoring by repeatedly scanning the local storage of these devices for new images/reports, by using DICOM query/retrieve or other network protocols. Alternatively, or in combination, in some embodiments, the on-site unit 105 may listen to HL7 network broadcast messages that may inform the on-site unit (and other systems in the hospital) of recently created patient images/reports and their location on the network. For more information on the autoburn feature, U.S. Pat. No. 7,302,164, the full disclosure of which is hereby incorporated by reference and is also attached hereto and made a part of this application.

In block 353, if auto-burn is configured on (it can be configured “on” on a per storage location basis (specific modalities, PACS, etc.), type of patient basis, or any other criteria), and the location of newly created images/reports is known by the on-site unit, the on-site unit may transfer the local images/reports to the on-site unit (and/or local FCS storage, etc.) 357 (for the purposes of the figure, these images/reports are considered “selected”). This transfer may be accomplished through DICOM query/retrieve, or any other protocol used to transfer images within a medical network.

In block 354, if auto-burn is not configured on, or if a medical facility's authorized user/administrator simply chooses to do so, a user or administrator may begin searching for files to create a manual CD/DVD (or other portable media) or upload packages with selected images/reports. In block 354, a user or administrator may contact a server to perform a search of imaging and report databases located at an MDR. This server may be located at the on-site unit or may be located in the cloud 102. This server may be configured with the location of one or more image and/or report databases (such as modalities, PACS, HIS, RIS, Mitra brokers, specific workstations, or any other device on a medical facilities network that can store images or reports).

For example, in some embodiments, the on-site unit may comprise a web server that implements a web interface. This web interface may have organizational users configured, similar to the cloud server described above (these users may be the same, as one skilled in the art would recognize, via using remote database calls or a syncing mechanism). After an authorized user logs into this interface, the user may search the configured databases described above based on a variety of search criteria, including patient name, patient id, patient's time period for stay or procedure, type of image or report, and the repository to be searched, among others. Alternatively, or in combination, the cloud server 102, through its web interface, may offer the same searching capabilities to search an organization's image and/or report databases.

In block 355, based on the search criteria, the server may query the configured or selected databases, using a DICOM protocol, or other protocol such as HTTP, HL7, SQL query, or other application layer protocol. Each of these databases may return a list of results that match the search criteria. The server may display via a user interface some or all of the results, or a summary of the results. In block 356, a user may select one, some, or all of the results (for example, can select all results for a single patient or multiple patients, or single studies for a patient, etc., or any combination thereof.)

In block 357, the selected DICOM images and/or reports can then be transferred from the databases containing those images and/or reports to the server. Thus, going into blocks 358 and later, whether using the auto-burn feature or selecting images manually, there is now a group of images that the system is working with to export (either via portable media CD/DVD or sent to the cloud).

In some embodiments, the server/on-site unit may perform a search for related data to the data selected 358. The server may have configured a list of databases (similar or the same as the list of databases to search initially) with images and/or reports to search for information related to the selected information. The system may be configured to automatically search these databases for related images/reports, or in other embodiments, a user may choose whether or not to perform a search for related images/reports. If related images/reports are found, these images/reports may be incorporated into the set of selected data to be exported by the server. After identifying the related data, the related data can be transferred to the server via DICOM query/retrieve, HL7, or any other application layer protocol. Additional methods to perform a search for related data can be found in U.S. Pat. No. 7,302,164.

In block 360, via the user interface, the user may decide whether to export the data via a CD/DVD (or other portable media) DICOM disk, or whether to upload the selected data as a virtual package. Such an indication is received by the server, usually via an HTTP post or GET parameter based on a web form but may be any electronic indicator that is transferred from the user's workstation and/or browser to the server/on-site unit.

If CD/DVD or portable media is selected, the on-site unit will assemble, based on the images/reports and accompanying DICOM data (such as patient name, patient id, accession number, etc.), CD-ROM images to be burned to a CD/DVD (or stored on other portable media) 362. For example, the on-site unit may create a DICOMDIR structure that identifies all of the DICOM studies (with contain images and/or reports) to be stored on a single disc. The system then creates a CD/DVD images with all of the selected data, including corresponding DICOM data and structure, selected images and reports (and discovered related data if applicable). The system may extract information from the DICOM meta information about the patient, studies or procedure to print as labels on the CD/DVD. This information may also be extracted in other ways, such as from stored HL7 broadcast messages, queries to a HIS or RIS, or any other database within the medical facility. Based on the size of the data to be written to the media, one or more jobs may be created if large enough to span more than one CD/DVD. After the jobs are created, and the label information determined, this information can be sent to the on-site unit for service. The CD/DVD images can be used by the on-site unit to burn the requested CD/DVD. The label information can be fed to the on-site unit's label printer, or a separate labeling or printing system. Once the disc is created, it can be sent to an intended recipient (whose contact information may be recorded on the label as well from user input during the process or by extracting it from stored data about an intended recipient).

In some embodiments, a user interacting with the server may choose instead to upload the collection of DICOM studies containing patient images and/or reports to the cloud system 359. This involves packaging together, in a virtual package, all of the various medical data including DICOM metadata, images, and reports. All of this data may be uploaded to the cloud system by the server via a DICOM transfer, or by direct storage into the cloud database via a CAS or FCS storage request (or by using methods discussed later for data upload to the cloud).

The user may have the option to specify whether this transfer to the cloud is temporary, or permanent, or whether it is for transfer (sending to another user or recipient) or backup purposes. If temporary, the user may be able to specify the time period (length of days, months, etc.) that the data should be kept by the cloud system for use. Similarly, if a user selects that the images are being uploaded to the cloud for transfer services, the data might be automatically set to be stored for a limited number of days (or some limited time period), whereas if kept for backup purpose, the data may be kept longer or indefinitely. For example, the cloud service may be instructed to store the uploaded data temporarily (which can indicate, for example, 1, 2, 5, 10, 20, 30, 50, 100, 200 days, etc.), permanently (which can indicate a long period of days, such as 365, 999, etc., or indefinite storage), or for a user specified number of days.

Similarly, in some embodiments, as a part of the upload process, a user may be able to select whether the upload is intended for a specific recipient. The user may identify one or more recipients to be able to access the data in the cloud by entering an email address of any recipient, or by instructing the server to search the cloud database or local database for a specific recipient by name, patient id, organization, or any other criteria collected by the server or cloud server about a recipient (note, the cloud server and the server can possibly be the same server). For example, after determining that the selected medical data will become part of a virtual package to be uploaded to the cloud, the user may type in an email address to the interface to indicate that, after upload to the cloud, these images should be made available the user associated with the typed in email address.

A virtual package, once the underlying data is recorded and stored by the cloud database, may be a group if identifiers that refer to each individual element of a package that is stored separately by the cloud system. For example, each study uploaded to the cloud may be stored separately in the cloud database 103 or together in one file. In some embodiments, each study may be stored within an FCS or CAS as a single file, and the virtual package is a collection of file “tickets” that can be used to read these stored files. U.S. Patent Publication 2012/0005252 describes a virtual package in more detail, and this publication's disclosure is fully incorporated by reference and is also attached hereto and made a part of this application. Once uploaded, metadata may be stored in the cloud database 102 about each individual study within a virtual package, and/or each virtual package itself. Such metadata may include the patient name, patient id, accession number, a any assign tracking number created by the cloud system for a study or virtual package, whether any image notifications have been sent to the recipient, the date the study was taken, the modality type, a description of the study, what facility or organization it came from or is affiliated with, a date or time to purge the associated file, the date the study was uploaded to the cloud, etc. This metadata may be extracted or calculated from the upload by the server, which may include either the cloud server and/or on-site unit.

In block 361, if images/reports are chosen to be uploaded to the cloud, an email may be sent by the cloud server to any recipient's email address. This email address may provide a URL link to access the images stored within the cloud. To access the images/reports, a recipient may click on the link which would launch a web browser that accesses the cloud server 102. In some embodiments, no logon may be required--access to the link is enough to guarantee access. In other embodiments, the recipient may have to be registered already, as described above, and enter their authentication credentials to gain access to login to the cloud server and gain access to their images. If the user is not registered, the URL link may be treated as an invitation to register as a recipient, who must be approved/validated to view images. In that case, sending the images may follow the normal recipient registration process described above. Alternatively, a separate URL may also be sent in the email (or a separate email) so that allows a recipient to begin the registration process with the cloud server.

### Methods and Systems for Uploading Data to Cloud

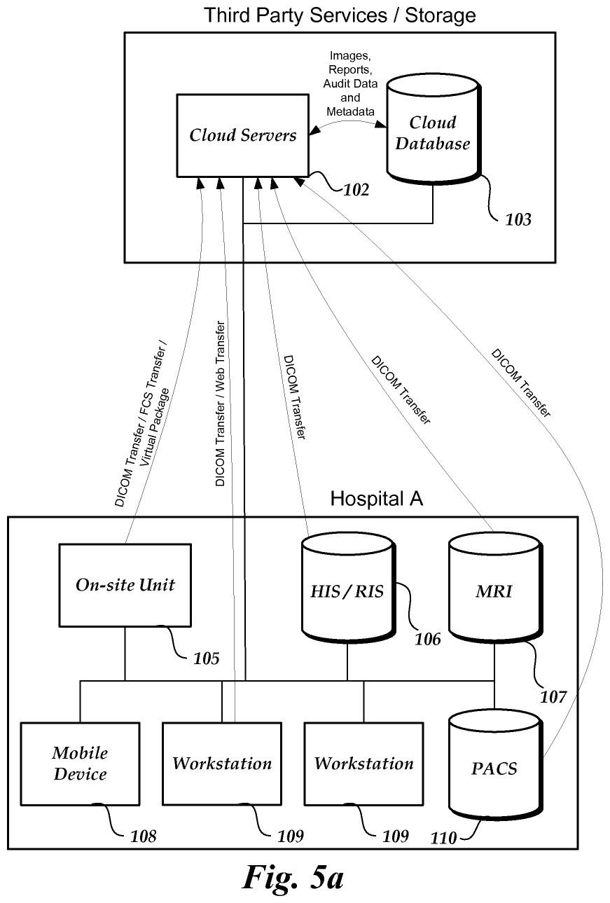

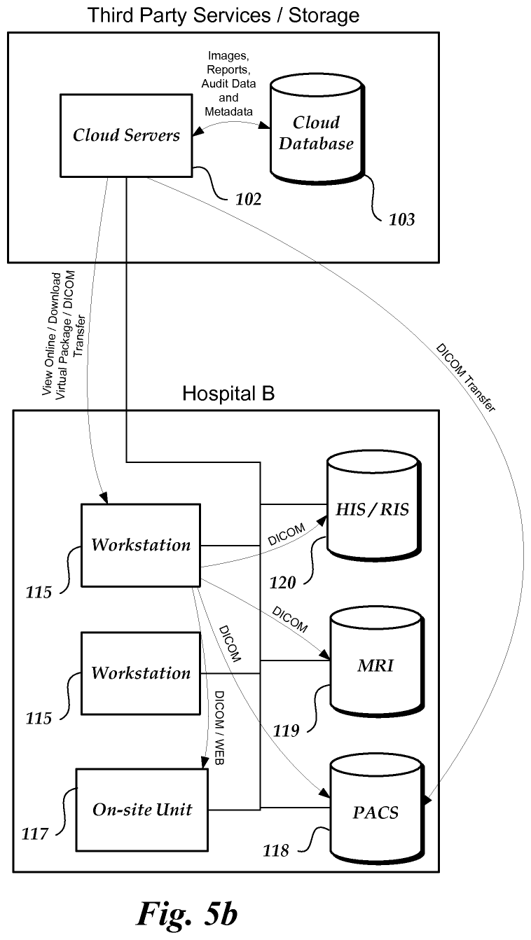

Alone, or in combination with the workflow described above for storing images and/or reports in the cloud, a user or organization may transfer data for storage in the cloud (or to be sent to another recipient/cloud user) in a variety of ways. FIG. 5a describes how example hospital A might send images and/or reports to the cloud.

In some embodiments, individual studies or virtual packages can be sent to the cloud by accessing a web server associated with the on-site unit (such as the PACSCube). By searching and selecting the studies to be sent to the cloud, by for example, following the example process in FIG. 3c, the on-site unit may upload the contents of images/reports and accompanying metadata (such as recipients or any DICOM header information) to the cloud servers for storage in the cloud database.

Such an upload may occur by sending one or more DICOM transfers to an IP address associated with the cloud servers. For example, the onsite unit may implement a DICOM Service Class User (SCU) that can send a DICOM C-STORE operation to the cloud server that is acting as a DICOM Service Class Provider. This operation will send the DICOM files to the cloud server which can then parse the DICOM files accordingly, store them, and store parsed and extracted metadata about them. If the on-site unit is acting as a part of the cloud database's fixed content storage (FCS) system, then it is also possible to use native FCS commands to store the files to the cloud server. In this case, the onsite unit would initiate a transfer or store command to the cloud's FCS database for the files it chose to store. Other methods of storing the DICOM files into the cloud include FTP, HTTP, or NFS.

Additional data can be sent from the onsite unit to the cloud server other than the DICOM files stored through DICOM or native FCS (such as a list of recipients for the files or a list of studies that comprise a virtual package). In this case an additional transfer channel may be used, which can include a web service (or any other HTTP protocol transfer) that is run by the cloud server and accessed by the onsite unit to transfer this information. Other options for this transfer include NFS and FTP transfers. Alternatively, or in combination, the system may also support the onsite unit transferring all of the DICOM and metadata to the cloud server, for parsing and storage in the cloud database, via an upload using HTTP.

Such transfers may occur in serial or parallel. For example, some optimization may occur for upload times if more than one DICOM study or series is uploaded at a time from the onsite unit to the cloud. This may be accomplished by opening multiple HTTP or DICOM connections to the cloud.

Workstations, such as 109 in FIG. 5a, may also upload directly to the cloud servers. Although a workstation might access a web server on the onsite unit to perform the upload, as described in FIG. 3c, a workstation might also choose to import DICOM studies to the cloud that the workstation has access to (either internally in its permanent storage, or portable storage (e.g. on a DICOM CD/DVD of patient images/reports), or various PACS and networked databases at the medical facility). For example, the workstation may execute software that may import DICOM files from these locations into the cloud. This software may read the DICOM files to be sent to the cloud system, and query the user to enter certain metadata, including any alterations to the DICOM tags for the uploaded files. DICOM tags for the uploaded DICOM images/reports may be desired because the DICOM files may have been created by a different medical facility than where the workstation is located. In that case, the DICOM metadata, such as patient ID, may not match the patient ID of the organization that wants to upload the files to the cloud. Thus, the software may query the user for changes to these fields so that before the files are uploaded to the cloud, the DICOM fields will be modified with the data desired by the user, and these updates will be reflected in the cloud. The software may automatically determine, or assist the user in determining, the correct entries for these values by searching local PACS, HIS or MS databases, and automatically replacing these values and/or suggesting values, including suggesting possible matching internal patients that a user can pick from to user their values. These DICOM files may be uploaded to the cloud directly via a DICOM send, as detailed above in discussion about the onsite unit performing such a transfer. In addition, a DICOM transfer to the cloud may be required to be accompanied by a web login to the cloud server by an authorized user to prevent unauthorized uploads from occurring. Similar to a workstation, a HIS, RIS, Modality or PACS system can upload directly to the cloud server using the same methods described for a workstation.

### Using and Downloading Images

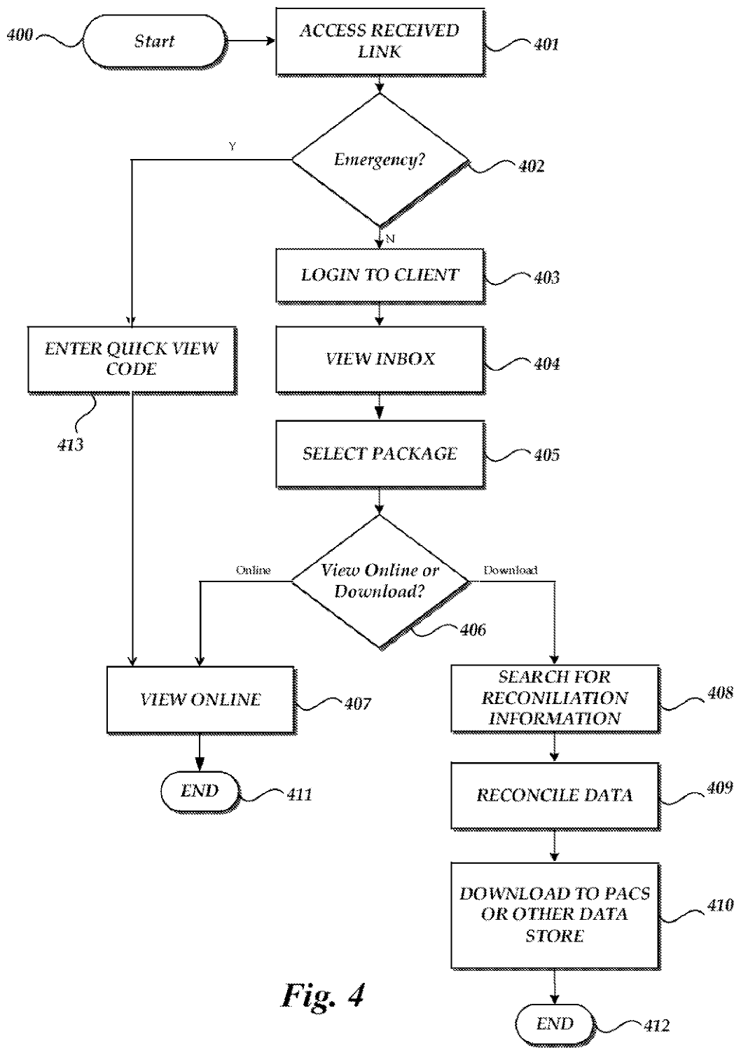

Referring to FIG. 4, in some embodiments, once data is uploaded to the cloud, a user may receive a link indicating that there are images/reports that have been sent to them for viewing. It must be noted that this is not the only embodiment. Images and reports can be uploaded to the cloud without specifying a specific recipient. In this case, those images are available for viewing in an inbox corresponding to the user and/or organization that uploaded them to the cloud.

The link received in block 401 may point a browser to a variety of locations, including a direct link to the images on the cloud system, a link to a registered user's inbox, a link to the registration system for unregistered users, etc. These links may appear alone or together with other directed links in the email. Regardless of what type of link is sent, a user after accessing the link will be required to either register as a recipient, or, if already registered, to login to the cloud server.

Referring to block 402, in some embodiments, images may be sent to a recipient as a part of a medical emergency. For example, images may be sent to an expert doctor away on vacation to diagnose a specific ailment that requires immediate attention. The expert doctor may not have a registration with the cloud service or be validated in the system by the organization administrator. Nevertheless, if an image upload by a user at an organization indicates that the uploaded images/reports about a patient have been uploaded as a part of an emergency, then a user accessing the link may view and download the images without logging in or registering in block 407. The images can be viewed using the online viewer discussed below.

In some embodiments, a quick view code may be used to regulate emergency access. A quick view code is simply a string of characters of any length (e.g. 4, 6, 8, 10, 20 characters, etc. such as Hnj86&54) that can be transmitted along with the email link, in a separate email, or by SMS text, or other communication method. The cloud service may require the code to be communicated to the cloud service or entered into its web interface before any access to the images are allowed. In this manner, access to the images is still secure and restricted without the medical provider having to go through the step of registering for the cloud service, which may cost precious time during an emergency.

In block 403, if the cloud system did not receive any indication from the controlling user or organization that the images have been sent to the recipient as part of an emergency, the user may login to the cloud servers after following the link (or alternatively complete the registration process and then login to the system if the recipient or organization user didn't exist.) Once logged in, the link may have sent the user/recipient directly to a set of images. This may be accomplished by the cloud server remembering the specific link URL the user clicked on and forwarding the user back to this URL after login is complete. This can be considered automatically selecting a package or study to view in 405, which can then be downloaded or viewed based on the decision in 406.

However, if the link was not to a specific set (i.e. study or virtual package) of images/reports, or if the user simply logged on to the system as a normal user or recipient without using an email link, for example, by simply opening their browser and navigating to the cloud server's website, the cloud server may present the user/recipient with a listing of all studies and/or packages. In some embodiments, the studies and/or packages listed in the inbox may be all (or a portion thereof) of an organizations' uploaded studies and packages, or may be all (or a portion thereof) of studies and/or packages that a user or recipient has rights to access or was sent to them. The listed studies and/or packages may also be sorted by a variety of criteria, including package ID, study ID, patient ID, patient name, date of study or package, the modality of a study, study description, name of organization or medical facility controlling the medical images/reports, the amount of time the package or study will be stored in the cloud, etc.

Using the list of packages and/or studies, or by searching for specific packages and/or studies available to the user/recipient on various criteria (e.g. by patient name, patient id, etc.), a user/recipient may select a package or study 405 for viewing or for downloading 406.

If viewing, the user/recipient may view the images/reports within the selected study(s) or package. Such viewing may involve the browser downloading a viewing program that may run within (or outside) a web browser. For example, the viewing tool may be a Java, Adobe Flash or MS Silverlight application that may be executed within the browser. The viewing tool and/or browser may download in serial or in parallel, the packages, studies, series, images, and reports that are within the selected package/study to be viewed. Parallel downloading may decrease the time before analysis of the images/reports and diagnosis of the patient can occur. The viewing tool may have a partial set, or a full set, of DICOM image and report viewing features, including the ability to view MRI, CT, PET, Ultrasounds, movies, among other types of images. The viewing tool may download lossy or lossless images and may be able to support viewing 2d and 3d images. It may be able to do normal graphical manipulations found in normal PACS viewers, such as zoom, rotate and markup of the images or reports, etc.

If downloading (or if choosing to download the images while within the viewing tool), the selected packages and/or studies may be downloaded to the local machine of the user/recipient accessing the cloud server. Before or after downloading the package and/or studies, the cloud server, or computer downloading the data may attempt to reconcile the DICOM tags of the cloud version of the images (usually from the uploading organization) with the appropriate information from the downloader's organization. For example, if Hospital A sent images/reports via the cloud service to a recipient at hospital B, before or after the recipient downloads the images, the DICOM headers within the file may be changed so that the patient ID or patient name (or other pertinent data within the DICOM headers) is updated with the information of hospital B. For example, if the DICOM headers of a DICOM study created at hospital A use a patient name of “John Doe” with patient id “837858”, the downloaded version may be translated to use patient name “John Q. Doe” with patient id “927654” that the same patient uses at hospital B.

For example, the cloud server or the computer downloading the data may query the user to enter certain metadata, including any alterations to the DICOM tags for the downloaded files. The cloud server or the downloading computer may query the user for changes to these fields so that before or after the files are downloaded from the cloud, the DICOM fields will be modified with the data desired by the user, and these updates will be reflected in the downloaded version. The software may automatically determine, or assist the user in determining, the correct entries for these values by searching local PACS, modality worklist, HIS or MS databases 408, and automatically replacing these values and/or suggesting values, including suggesting possible matching internal patients that a user can pick from to user their values 409. For example, the downloading computer or the cloud server may have stored a list of configured PACS, modality worklist, HIS, MS or other databases affiliated with the recipient or the recipient organization that can be contacted to search for matching patient information. If a match is found, the DICOM headers may be automatically translated with the patient data from the most relevant search result, or specific search result entries can be selected by the user/recipient to use as a patient data template for translation. The cloud server or the downloading computer may then replace the hospital specific DICOM header data with the selected search result entry's identification data.

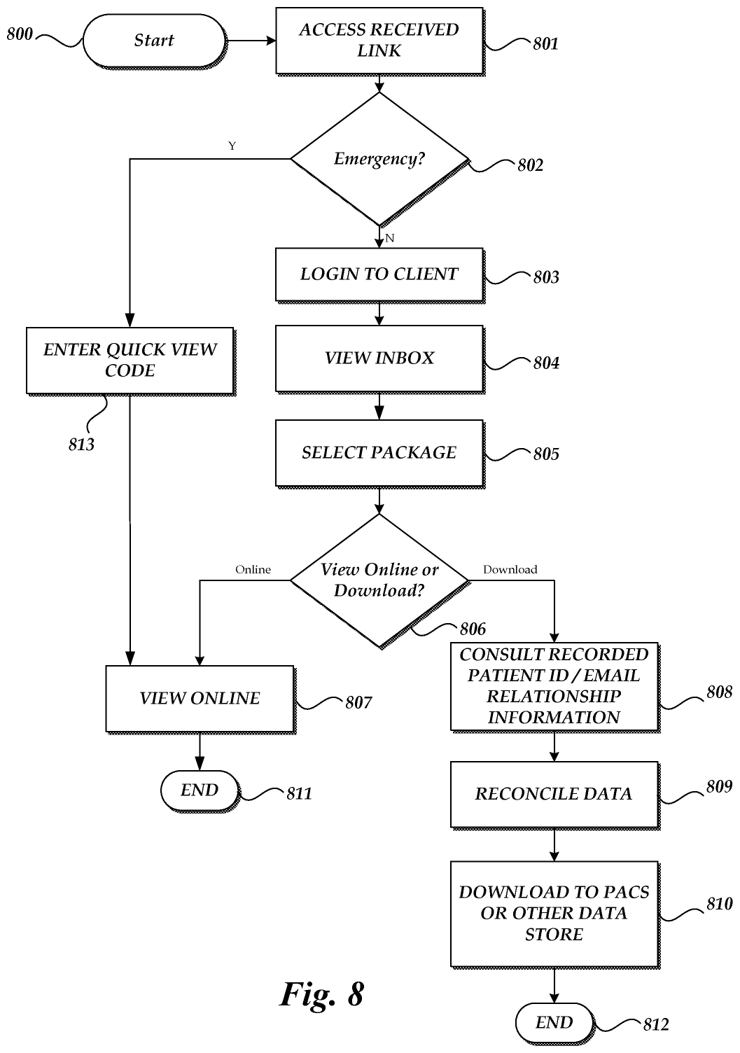

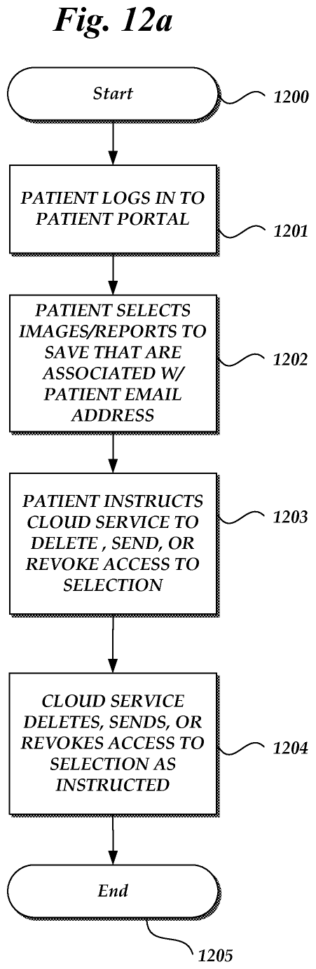

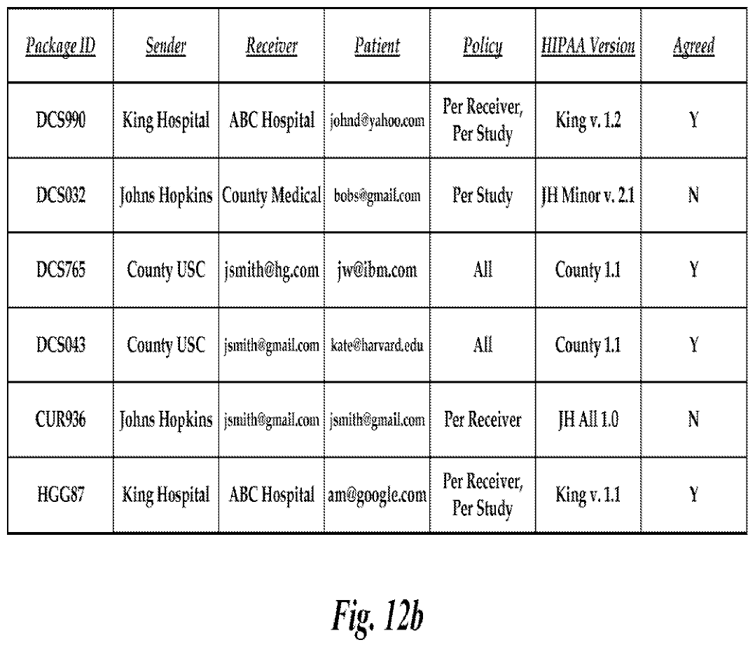

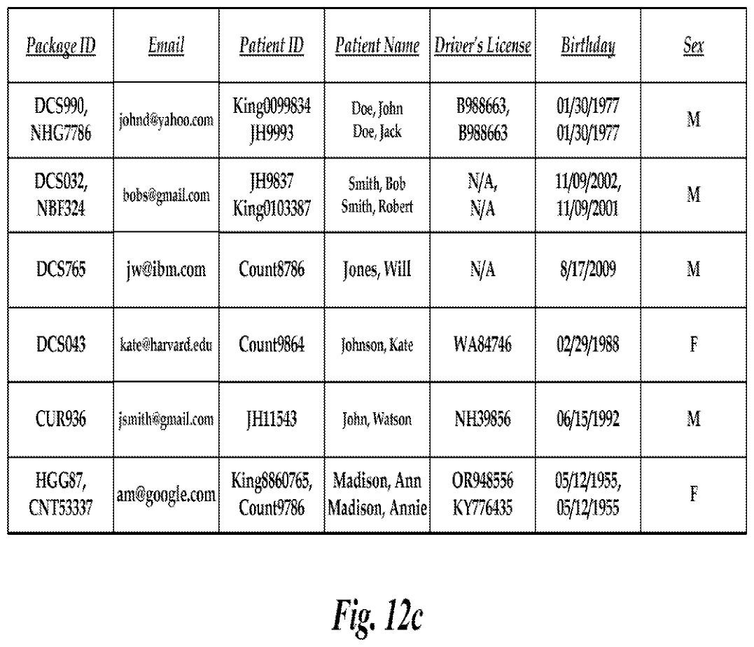

In one embodiment, as shown in FIG. 8, a table or tables, such as the one shown in FIG. 12c may be consulted to automatically translate patient IDs, patient names, or other patient related information between institutions. The creation of this table in FIG. 12c in the cloud service's data store may occur through the use of parsing DICOM information from packages/studies and through the collection of the HIPAA releases as described in the HIPAA section. The process may search the table by looking for matching patient IDs that correspond with the sending institution. If a match is found, and the data store contains patient ID or other matching information about that patient for the receiving institution, translation may occur, where the DICOM headers or any other metadata about that patient may be replaced with the different information for the receiving institution. For example, referring to FIG. 12c, a DICOM image sent from King Hospital to Johns Hopkins about patient John Doe may contain the patient ID King0099834 and Patient Name “John Doe”. The system may automatically alter the information downloaded by Johns Hopkins on the fly so that all images will instead contain the patient ID 1119993 and the name Jack Doe in the DICOM headers, as reflected in the table row. In this manner, the system may automatically reconfigure the downloaded data so that it is compatible with Johns Hopkins IT system.

In block 410, one package and/or studies have been downloaded, the downloading computer may transfer them to a PACS or other database at a facility via a DICOM transfer, or any other mechanism. In some embodiments, the data can be downloaded directly to a PACS or other database, and now the downloading computer. This can be achieved via DICOM transfer from the cloud server directly to the PACS. The user/recipient's computer may instruct the cloud server to download it to the PACS and may supply the PACS's (or any other facility database's) network addressing information. In some embodiments, the cloud server is aware of the PACS or other medical database at the facility and may transfer the package/studies directly to one or more of these databases without specification by the user.

For example, referring to FIG. 5b, a workstation can be used to download packages and/or studies from the cloud server 103. As describe above, the images may be viewed on the workstation through the browser or downloaded to the workstation. This transfer may occur via HTTP, HTTPS, DICOM, or any other transfer mechanism. Once downloaded (and DICOM headers possibly reconciled), the workstation may transfer the DICOM study(s) to various databases at the medical facility, including a HIS or MS 120, a modality, a PACS 118, or the onsite unit 117, among others. In some embodiments, the cloud server may be instructed by a recipient or user to transfer directly from the cloud server via a DICOM transfer to a database at the facility, such as PACS 118.

### Requiring HIPAA Releases and the HIPAA Release Process

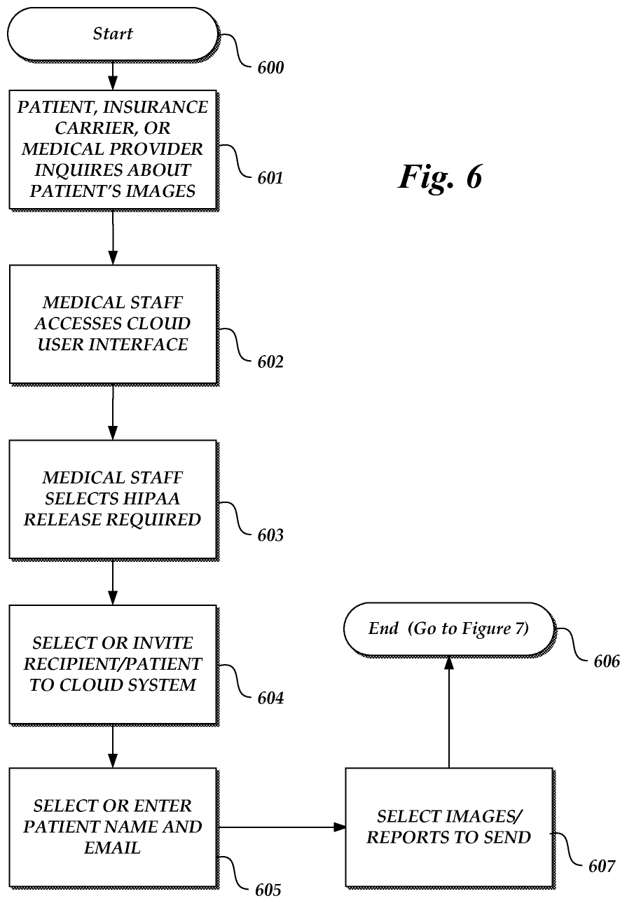

Turning to FIG. 6, a patient or organization interested in accessing an MDR's medical information about a patient may request the transfer of the patient's images, medical reports, and other medical data such as an electronic medical record to their own institution's IT infrastructure (601). For example, a patient, insurance carrier, or medical care provider may inquire about images for a certain patient. This may involve either calling on the phone to the MDR having the patient information or use of electronic means to send a request to the MDR. For example, in some embodiments, the third party cloud service can receive a request using its user interface for data about a particular patient for, by example, receiving the patient's name, id, or other identifying information in electronic form and either matching that data within its own record to determine whether the requested images exist on its own third party cloud service network, or send the request to the MDR where the patient's data is stored to handle the request.

When a request is made, HIPAA regulations often require that the patient associated with the medical data (or patent, guardian, or legal trustee making medical decisions about such patient) has legally released under the HIPAA guidelines the MDR to transmit the data to an organization, person, or entity outside of the MDR, its organization or affiliates.

When a request for patient information is submitted to the MDR, electronically or otherwise, medical staff at the MDR (or otherwise affiliated with the MDR) may access the cloud service's user interface to select (or upload) a specific HIPAA document that must be agreed to by the patient before the data may be sent.

Each electronic version of a HIPAA release may contain a variety of data and attributes that may be used by the cloud service to implement certain security features. For example, a HIPAA release may comprise both a legal data portion, and an attribute portion. The legal data portion may comprise up legal text. For example, the text of a contract or release that can be entered into between the patient and the MDR. This text may have a variety of restrictions, clauses or limitations. For example, the HIPAA release may only permit the MDR to release medical information to a specific recipient (specific person or hospital, insurance carrier, or other entity or organization). The attribute portion of the electronic HIPAA release may contain specific electronic information that may be used by the cloud service to enforce HIPAA compliance with an agreed to contract. For example, attributes for a HIPAA release may be the title of a release, the version of a release, whether the release allows transfer to all outside entities or just one (or a limited set) of outside entities, and whether the release applies to all DICOM studies of the patient or just a specific study or virtual package, among other criteria.

These electronic fields associated with the legal text of the HIPAA release enable the cloud service to enforce certain requirements of the HIPAA releases. Unless a HIPAA release was agreed to by the patient (or appropriate legal entity) that is the correct version required for the study, that applies to the particular entity to receive the study, and that applies to the particular study to be sent or all studies, the cloud service (or affiliated on-site unit) may not send the data to the cloud, and/or may not allow a receiver to download or view the data.

The step 603 medical staff selecting a particular HIPAA release required to be signed may be done automatically, or as a matter of policy or course. The cloud service may support allowing an administrator or account affiliated with an organization of appropriate role to set default requirements, or policies, to enforce when sending out medical data for patients. For example, the system may be configured to use a specific HIPAA release for all patients, or use different HIPAA releases depending on patient data such as patient type, age, location, etc. (such patient data may be read from DICOM fields or stored in the cloud service database). These defaults, policies may allow the cloud service or on-site unit to automatically select a HIPAA release to be used for transfer of medical data such as a DICOM study containing images and/or structured reports.

Once the medical staff, for example a nurse, doctor, orderly, or lawyer at the medical institution has selected the required HIPAA release using the cloud or on-site unit's user interface, or the HIPAA release was automatically selected, the medical staff may select the patient or recipient to receive the medical data (604). This may include adding a new recipient or patient to the cloud service system (and related on-site unit) or selecting the recipient if already included in the system. The user may add a recipient or patient, or select a recipient or patient by, for example, entering the patient's name and/or email address into the cloud service's (or on-site unit's) user interface (605). After the cloud service of on-site unit has received this selection, it may use this information to search the cloud service or PACS (or other local MDR data store such as a modality, HIS or RIS) and identify matching patients to present choices of patients to the user. This search may also involve returning various images, DICOM structured reports, other reports, or other medical data that can be selected by a user to send to a requestor (or without request) via upload to the cloud service.

In some embodiments, the order may be reversed. The patient/recipient selection and the images or other medical data to send selection may be performed prior to selecting the HIPAA release (or automatically selecting the HIPAA release). In addition, these actions may be performed by different devices facilitating the medical staff's selections. For example, selecting the HIPAA release may be performed by the cloud service, whereas selecting the patient may be performed by the user sending web or application data instructions to the on-site unit before uploading the data to the cloud.

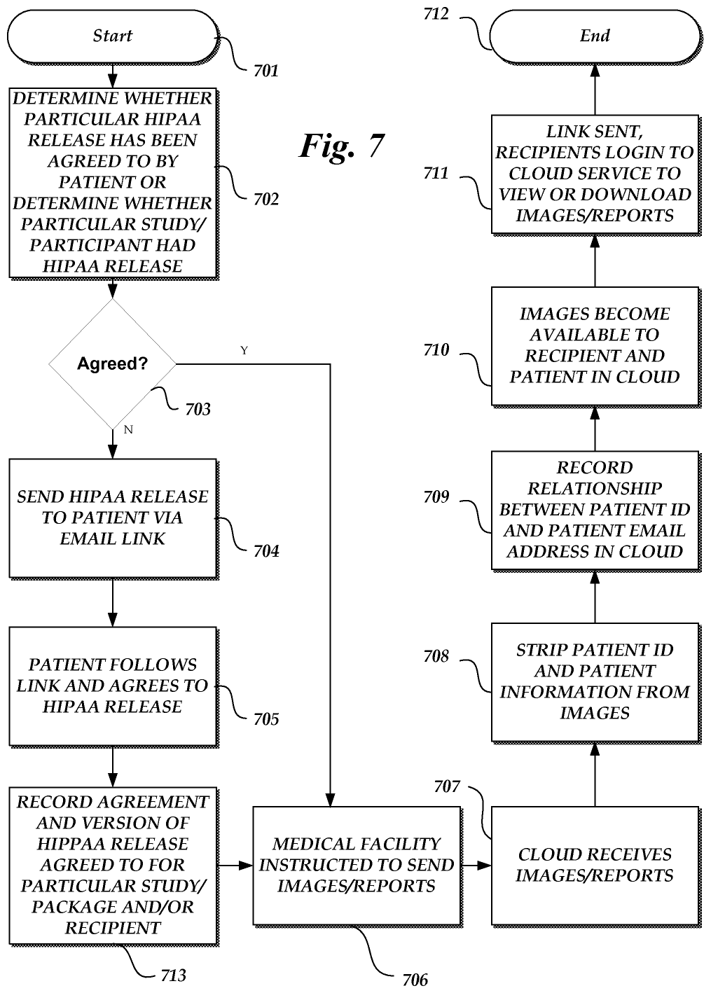

Turning to FIG. 7, in some embodiments, the HIPAA release need not be agreed to by the patient or their legal representatives every time a virtual package or study is going to be sent via the cloud service from an MDR. In some cases, the study to be sent may have already been sent to the same or a different institution. In that case, the HIPAA release may have already been agreed to, and that agreement may have been recorded in the cloud service's database. If the cloud service (or on-site unit) determines that the patient has already agreed to a sufficient HIPAA release 702, the system may bypass obtaining a patient's agreement before sending the medical data to the cloud service 706 (or release of access data already stored in the cloud service after transfer to the cloud service has already occurred).

For example, a cloud service may store records as shown in FIG. 12b, which represents one possible embodiment's table that tracks whether a particular study has a HIPAA release, what it applies to, and whether a patient has agreed to the release. Such a table could be implemented in an SQL database, or any other database or data store that allows for the storage, reading, and retrieval of organized information. This table is only representative and can be implemented in multiple ways including using multiple tables. In addition, the table could use other outside information also stored in a database. For example, the cloud system could also use a table for all the legal data and attributes of particular HIPAA release. In this example, some of those details are represented in this table but could easily exist in other tables of a different format.

In FIG. 12b, the study or studies with virtual Package ID DCS990 corresponds with studies that are to be sent “King Hospital” and received by “ABC Hospital”, regarding a patient with email address johnd@yahoo.com. The email address used to ID the patient is but one embodiment, and anything associated with the patient could be used to identify the patient, including email address, patient ID or name. To transmit the virtual package, the HIPAA Version column indicates that the “King v. 1.2” HIPAA release is required to be agreed to. The policy column indicates that this HIPAA release only releases patient information on a “per study”, and “per receiver” basis, meaning that the HIPAA release only applies to package DCS990 and when that package is sent to ABC Hospital alone. This allows for HIPAA releases to be determined on a granular level. For DCS990, the policy field means that, if a different package was to be sent about patient johnd@yahoo.com, even if to ABC Hospital, another release must be agreed to by the patient (per study). Additionally, even if the same package, DCS990, was to be sent to a different hospital, a new HIPAA release must be agreed to (per receiver). Looking at the row for DCS765 which uses “County 1.1” HIPAA release, the row indicates that the release applies to “All” recipients and all studies. Thus, if another package was to be sent regarding medical information on patient jw@ibm.com from County USC hospital, the patient would not need to agree to a new HIPAA release.

In this particular case, the DCS990 row indicates that the release was already agreed to by a patient, and the system may skip obtaining the release 707. If the patient had not yet agreed to the release, the row would have an indicated an “N” in the “Agreed” column. In this case the agreement must be collected from the patient or their legal representative.

Blocks 704-706 describe the steps necessary to obtain a release for a specific patient. In one embodiment, the specific HIPAA release may be sent in email to a patient. In another embodiment, a link may be sent in email to the patient. Other avenues of communication may be used to send the release, including SMS text messaging, phone call with voice recognition, by using a human call center, or collecting the release in person. For the human call center and in person options, a user must input manually into the user interface of the cloud system that the particular patient has agreed to the specific HIPAA release requested. This may involve the patient signing a physical copy of the document and uploading a scan of that document to the cloud system.

Electronic collection of the HIPAA release, for example by a release sent through email or via accessing a URL link sent via email (704 and 705), the patient may be required to electronically sign the HIPAA release. This may involve typing in the patient's name as a signature or using a cryptographic signature such as any form of public/private key encryption. The signature method can any known to those in the art for signing electronic information. The signature (not shown), and date of signature (not shown), and specific release signed may be stored within the table (or tables) shown in FIG. 12b, 713. The database may be updated to indicate that the release has been agreed to, for example by updating the “Agreed” field from “N” to “Y” 713, thus allowing the associated package/study(ies) to be transmitted and/or accessed on the cloud service 706.

Once the HIPAA release has been accomplished, upload of the data to the cloud service is required. The upload of the package/study data may occur as already discussed under FIGS. 1b, 3b, 3c, and 5a above, wherein the package information may be transferred via HTTP, encrypted HTTP, HTTPS or DICOM (or any other protocol that allows for data transfer to the cloud, and may be routed through the on-site unit (i.e. transferred to the on-site unit from the workstation/mobile device or PACS, and then transferred from the on-site unit to the cloud service) or independent of the on-site unit. The package may also be uploaded directly from a PACS or other data store at the MDR straight to the cloud service.

In block 708, the DICOM headers of images or reports within the package may be parsed and the patient name, patient id, birthday, etc. (meta data information about the patient) may be recorded by the cloud service, and entered into its database (for example, into a table similar to FIG. 12(c) and related tables).

In block 709, the relationship between the patient and email address can be recorded in the cloud service database. Block 709 can occur at any time once the patient ID is known to the cloud service along with a patient ID or name for a particular MDR. The cloud service may collect patient IDs, names, and patient email addresses in a number of different ways. In certain embodiments, a patient email address is collected when a user from an MDR types into the cloud service user interface (or on-site unit's UI) in order to obtain a HIPAA release. In this embodiment, before medical data can be sent to the cloud service, the MDR's user types in the patient's email address in order to send the HIPAA release to the patient's (or legal representative's) email address. Associated with this email address and HIPAA release is the package to be sent, which contains DICOM information in one or more studies related to the patient. The DICOM information contains DICOM headers, which may contain fields such as patient name, patient ID, access number, or any other identifying characteristic. This information can be extracted and stored along with the patient or representative's email address in a table (or tables), such as the one in shown in FIG. 12c. This same information may be collected using this process across multiple institutions, and thus multiple patient IDs or names may be collected per patient and stored in the cloud service's database.

For example, King hospital sends package DCS990 to another MDR. DCS990 in FIG. 12c could have identified as being associated with patient “johnd@yahoo.com” during the HIPAA release collection process. The DICOM information associated with that patient as contained within the DICOM headers of package DCS990 contained a patient name of John Doe, and a patient ID of King0099834. Package NGH7786, sent by Johns Hopkins to a different MDR, may have also used the email address johnd@yahoo.com during the HIPAA collection process. However, the DICOM information of package NHG7786 recorded the patient's name as “Jack Doe” instead of “John Doe”, and Johns Hopkins used a different patient ID for the patient, JH9993. Despite the differences between patient name and patient ID, the cloud system can recognize that the images concerned the same patient because of use of the same email address gathered through the HIPAA process.

Other IDs can be used to uniquely identify patients and record the associated Patient IDs and names used at the various MDRs by the cloud service. For example, an algorithm could allow the cloud service to match patients using an algorithm or heuristic with a certain degree of confidence using birthday, ID information (SSN, driver's license, etc.), patient Name, email address, sex, contact information, or any other information unique to a patient that would narrow the scope of potential mistakes when matching up packages sent to and associated with individual patients.

One advantage of using the HIPAA process to collect unique email information about the patient is that the cloud service can build a master table (or tables) containing a unique patient and their associated names, aliases and patient IDs at various hospitals. This information can then be used to, among other things, automatically reconcile DICOM header information when DICOM medical data is being transferred between institutions.

For example, assuming the table in 12c has already been populated with the information shown, when a package is to be sent from King Hospital to Johns Hopkins regarding patient John Doe, the cloud service may use the table to reconcile the information prior to download into Johns Hopkins' PACS (or other data store). In this example, a package with DICOM information for King00099834 about “John Doe” that is from King Hospital is uploaded to the cloud to be transferred to Johns Hopkins. When (or before) Johns Hopkins downloads the data, the package may automatically be altered so that the Patient ID is JH9993 and the patient's name Jack Doe, which matches John Hopkins records. This allows for translation between hospital patient IDs, names, birthdays, or any data that may be different between hospitals (either intentionally or by mistakes). This removes the necessary step of human intervention to search for the correct patient data in Johns Hopkins medical IT system and correct the patient IDs or other patient information.

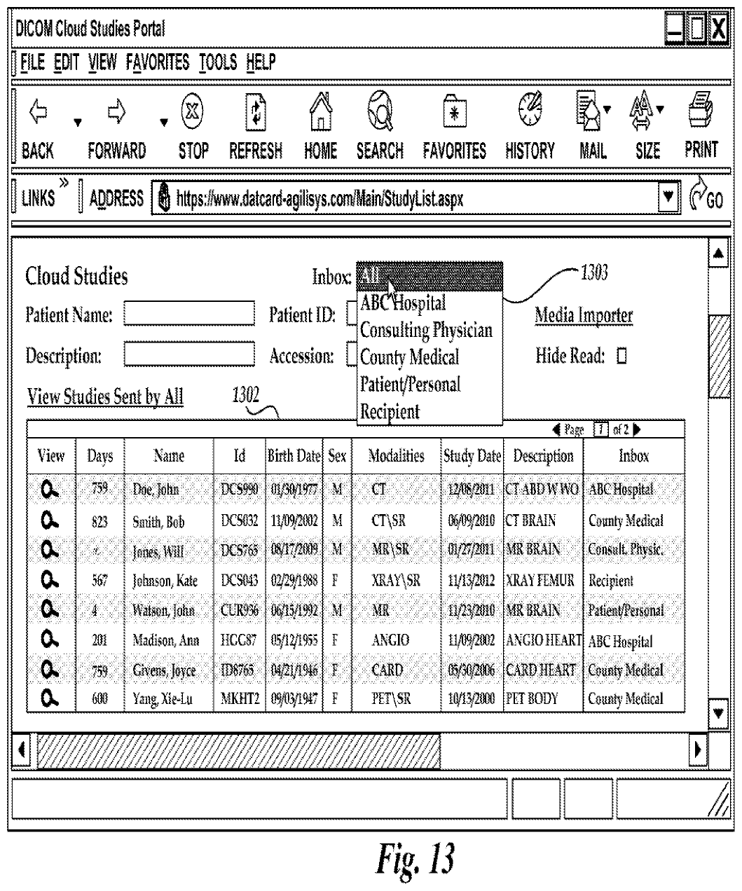

Returning to FIG. 7, the process proceeds to allow the images to become available to the recipient 710. This allows the package to be accessed through the recipients Inbox, as shown in FIG. 13, and discussed in detail in other sections. A link may be sent to an email address associated with the recipients to notify that the images/reports or other medical data are now available for download or viewing 711.

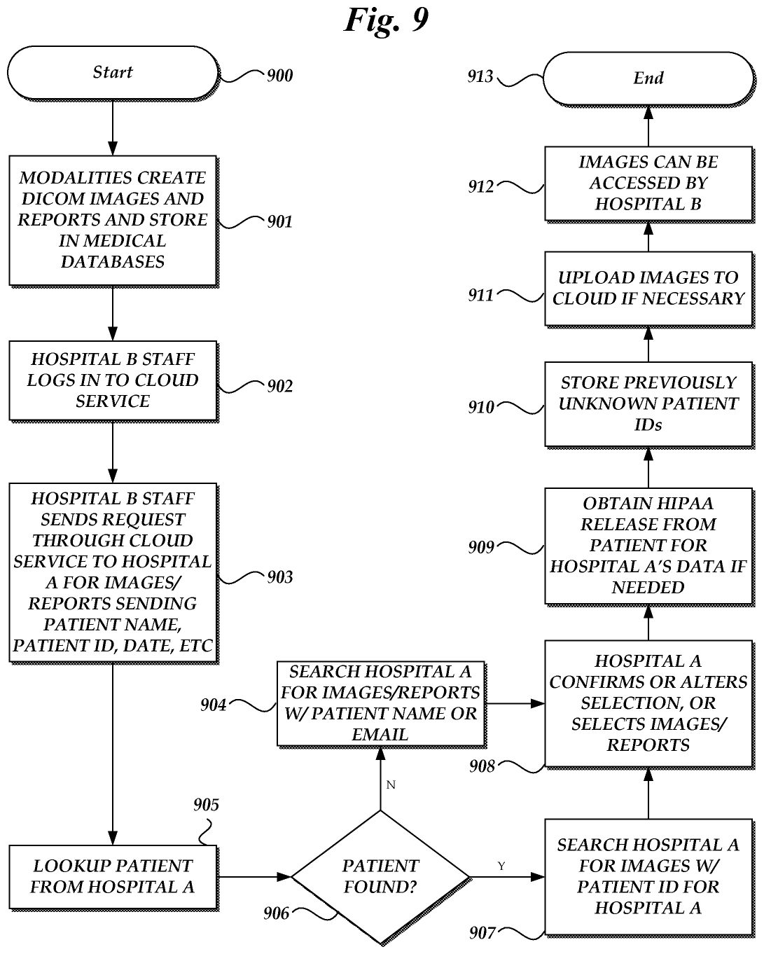

In some embodiments, the process may be altered as shown in FIG. 9 compared to FIG. 7. FIG. 9 depicts a process for requesting and sending images to the cloud with little human intervention. For example, in block 901, modalities at example Hospital A scan a patient (say, patient X) and create medical images for that patient. Those images may be stored in a PACS, on the modalities, or in any other medical image database.

In step 902, Hospital B desires the latest images for patient X. Staff from Hospital B logon to the cloud service to initiate the process to request images from Hospital A.

In 903, the cloud system can identify Hospital A as the hospital to gather this patient's records from. This can be accomplished by Hospital B′s staff already knowing that Hospital A has the images, and can perform text searching of the cloud service's data store to find Hospital A. Alternatively, staff at Hospital B may type in identifying information for Patient X (patient name, birthday, patient ID, sex, etc.). The cloud service may use this information to search its data store to determine what hospital the patient is at. It may perform this function by consulting example tables 12b and 12c (or any other patient to hospital correlation stored in the cloud service's data store). Once a hospital has been found, Hospital B may request images from Hospital A related to the patient. This request may be specific as to the images desired (for example, all chest xrays), or be generic in that it may convert all images for a patient.

As a part of the request process, this information may be forwarded to Hospital A to process 903. There are a number of embodiments for this scenario.

First, the on-site unit at Hospital A may receive the request for patient information. The request may comprise information identifying the patient that can be used to search Hospital A's medical IT infrastructure for the requested items (such as patient name, patient ID (translated using 12c or not), a date or date range identifying the images, the image type, the body portion depicted in the image, or any other information that allows identification of the desired image(s) or associated patent.

Using this information the on-site unit in Hospital A may search the hospital IT infrastructure for a particular patient 905 using DICOM, HL7 or any other appropriate protocol to locate the desired information. If the patient is found 906, the on-site unit may perform a search of data stores in the hospital (again using DICOM HL7, etc) for images (or reports, or other medical data depending on what is being requested) using the identified Patient ID at Hospital A. Alternatively the searches in 905 and 907 can be combined. For example, as an alternative, or if no patient was found in 906, Hospital A's medical IT infrastructure (PACS, modalities, HIS, RIS) can be searched for images/reports etc.

Regardless of what method is used, any search results found may be presented to staff at Hospital A 908. This allows for confirmation by Hospital A′s staff of the correct images, reports or medical data being sent to Hospital B as requested by Hospital B. In addition, it provides another security check to make sure that Hospital A is not sending out data to any other MDR that it does not want to release. At this stage, the staff at Hospital A have the opportunity to cancel the request/transaction and alter the set of images found so that the correct ones can be sent. Once complete, in blocks 909 through 912, an appropriate HIPAA release can be acquired from the patient and the images uploaded to the cloud service. These images can then be made available to Hospital B. These blocks are a more simple embodiment of the process that is described in FIG. 7 blocks 707-711, but either process portion may be used.

As an alternative embodiment, the on-site unit need not perform the searching of Hospital A for the patient data. Instead, that searching (and user interaction required in 908) may be performed by the cloud service itself. Upload of the images/reports may be performed by the workstation, or directly from a PACS or other data store in an MDR.

### HL7 Listener

The information described above that is collected during the HIPAA process and parsed from DICOM information uploaded to the cloud service may also be collected by the on-site unit at an MDR. The on-site unit need only to listen to HL7 protocol broadcast messages. These messages often include patient identification information, including full names, patient IDs, birthdays, sex, contact information, and medical history, among other information. This information may be parsed using standards set by the HL7 protocol and uploaded to populate the cloud database about specific patients at MDRs (for example, a table similar to table 12(c)).

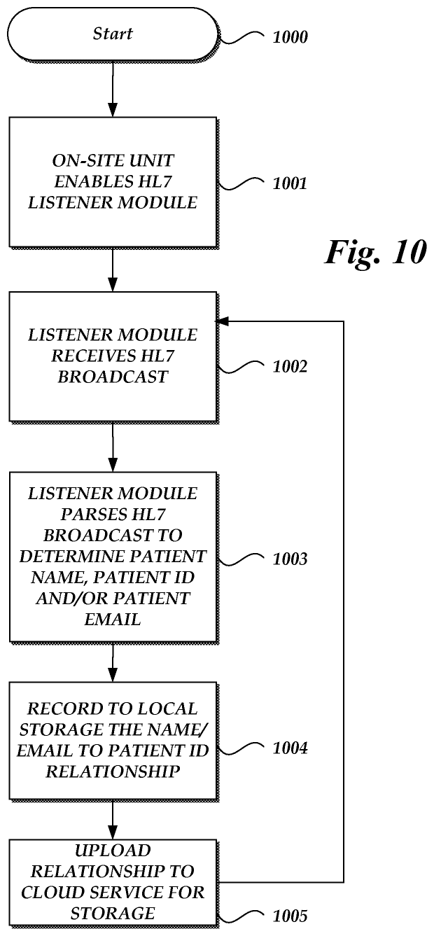

One potential embodiment for this functionality is depicted in FIG. 10. The on-site unit enables an HL7 listening module 1001. This listens to HL7 broadcast network traffic received on an MDR's network. An on-site unit in this scenario is deployed behind any firewall and has network connectivity to the MDR's IT infrastructure.

In step 1002, the listen module receives a new broadcast message. In step 1003, the listener module may then parse the received HL7 message into its fields and component parts in order to determine the patient's information such as name, patient ID, or patient email. In the preferred embodiment, the patient name and the patient email address can be identified.

In step 1004, the parsed patient information can be recorded locally onto the on-site unit's data store, including the patient ID and patient email address (along with any other pertinent patient information, such as birthday, sex, contact information, aliases, medical history, etc.).

Then, in step 1004, this information can be uploaded to the cloud and stored in the cloud service's database in a table(s) similar to table 12(c). This allows the cloud service to keep track of patients of MDRs in real time and pre-populate the email to patient ID relationship that is normally gathered through the HIPAA release process. One advantage of the HL7 listener module is that it is not dependent on the HIPAA release process but can still uniquely identify a patient between MDRs based on email address, and provide the data required to do such a reconciliation and translation for data travelling between MDRs. As an alternative, patient information learned by the listener module may be uploaded in batch to the cloud service.

### Patient Portal and Patient Option to Store Medical Information Indefinitely

In some embodiments, patients may have control and access over their own data. To interface with the cloud service, patients may be allowed to login as a normal or special recipient that can view, send, and remove images, and may approve access to their images. This interface may also be use in some embodiments to collect HIPAA releases from a patient when an outside medical provider or coverage/insurance entity requires or desires access to a patient's images. These services may be provided using a similar web browser interface describe above. The cloud service may provide this web service within the same DNS domain as for normal recipients and/or organizational users or may have a separate domain name that is used for patients to contact and log in to that is distinct from the organizational or recipient DNS domains.

A patient portal may provide at least two advantages. First, the portal assists in compliance of HIPAA laws and ensures a patient's medical data security. Unless a patient consents through the portal to an electronic HIPAA release prepared by an MDR, the MDR will not send any images to the cloud service, or to any other recipients. Second, if and when a patient does consent to a HIPAA release, the portal requires either the MDR or the patient to enter identifying information about the patient. This information, including for example the patient's email address that is used for communication with the patient, can be used to reconcile that patient's data cross multiple MDRs. For example, two different MDRs may use different patient IDs for the same patient. However, the cloud service can detect that the different patient IDs correspond to only one physical patient because the patient will typically use only one email address to interact with the cloud service. Thus, the cloud service may associate a single email address with multiple patient IDs across MDRs and may be able to reconcile data from disparate MDRs using different patient IDs using this association.

In some embodiments, the patient information may be stored only temporarily. FIG. 13 displays a number of remaining “Days” that particular packages that contain DICOM images and structured reports, and/or other medical data in the cloud service's database. However, once the data is in the cloud, a patient may be notified that their data is being held in the cloud. This can be accomplished by sending another link to the patient to their images and asking the patient to sign up with an account for the cloud service.

Once the patient has access to the cloud, and can download, remove access to, delete or view their medical information, it may also be advantageous for a patient to purchase permanent storage of their images. This would enable a patient to store the images permanently so that they are always available for their medical history.

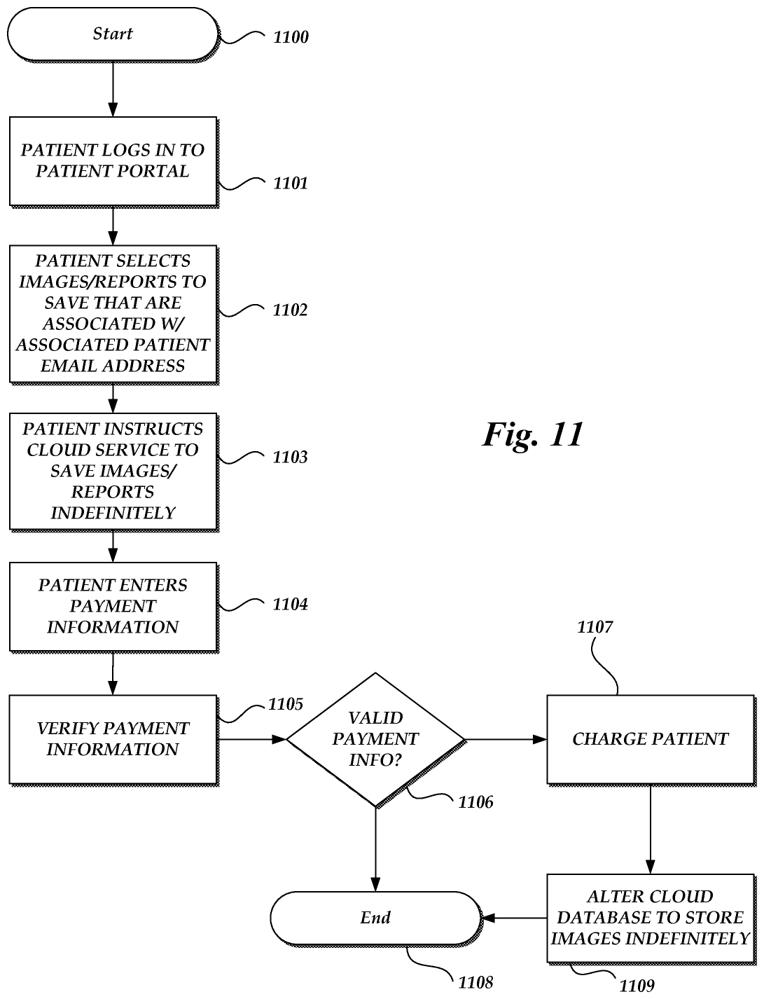

FIG. 11 depicts a process that allows a patient to request and pay for permanent storage of their uploaded medical information. In step 1101, the patient logs into the cloud service using the account they created. The user interface they are presented with may be customized for patients only. For example, it may have a reduced feature set compared to other users of the cloud service from MDRs. This reduced feature set may be considered the “patient portal”.

Once they have logged in an accessed the cloud service, in some embodiments, the patient may select the virtual package/study (i.e. image and reports) to save that they have access to, based on their email address 1102. The patient then sends an instruction to the cloud service to save the reports indefinitely 1103.

If the cloud service wishes to charge for this service, it may ask the patient to enter payment information, such as a credit card number with expiration date and security identifier or redirect to a payment site such as PayPal. After the user enters their payment information, the payment information is verified by querying a payment service such as a credit card processing service with an associated charge 1105. If successful 1106, the patient is charged and the cloud database is altered to reflect that the desired virtual package may be stored indefinitely (or for some other period of time by recording the period of time during which the study will not be deleted, which is configurable by the cloud service administrators, e.g. 100 days, 1000 days, 12 months, 24 months, etc.).

In some embodiments, patients may be able to upload medical information to the cloud provider for storage of the patient's medical data. A similar credit card verification and charge may be required. For one such example of how an upload may work, patent application publication 2011/0301981 by Duma et al. is incorporated by reference and is also attached hereto as an appendix and made a part of this application. In the Duma reference, patients may upload their medical information into a remote database server. Also related is patent application publication 2012/0179908 by Duma which is hereby incorporated by reference and is also attached hereto and made a part of this application.

### Security

Some embodiments may enlist a variety of security mechanisms to assist in reducing access to medical information stored within a facility or the cloud database by unauthorized users.

In some embodiments, a user or recipient password may be required by the cloud server to be a certain strength. For example, an administrator may select a password policy for his users, or all recipients added by his organization. This may be a requirement for any combination of non-words, lower case letters, capital letters, numbers, symbols, etc. In addition, the passwords stored in the cloud database may be salted, hashed, and/or encrypted so that any security breach that occurs of the cloud server does not reveal the passwords for all users of the cloud server's service. Additionally, when a user types in their password incorrectly multiple times, a user may be denied from logging in until an administrator resets the account to prevent unauthorized connections from guessing a user's password. Some embodiments may also inform a user or recipient of the IP address, location, or time of a previous login by their account (or a complete history of such logins), so that the user may determine if any unauthorized access occurred using their account.

When a user or recipient is logged into the cloud service, for example via a browser, the cloud server may track user/recipient activity by determining the amount of time that passes since the user/recipients last interaction. This can be a value recorded on a per user basis or tracked as a part of the auditing process. If idle time exceeds a certain threshold, the user or recipient can be considered logged out and must re-login once again to gain access to the cloud service.

Transfer of the actual images and reports in the studies and packages between the cloud server, cloud database, and databases at the medical facilities can be encrypted to protect confidentiality. Encryption mechanisms may include symmetric encryption, such as AES, or public private key encryption. Symmetric keys can be established ahead of time between organizations, users and the cloud service upon registration of the organization or user. Alternatively, symmetric keys can be negotiated over HTTPS, or any other public/private key exchange mechanism that allows for confidential symmetric key negotiation. Typically, access to the user interface of the cloud service through a web interface may be encrypted using standard SSL/TLS. In some embodiments, any transfer of data between a browser and the onsite unit may also be protected via SSL/TLS, and DICOM or normal HTTP transfer between the onsite unit and the cloud or local facility databases may be similarly symmetrically encrypted. Likewise, transfer of any traffic between a client workstation/mobile device and the cloud may be similarly encrypted using SSL/TLS, encrypted DICOM, or encrypting HTTP traffic, among other encryption options.

Patients may also have control over the data stored in the cloud service. As a part of the patient portal, a patient may be able to revoke access to particular information stored in the cloud. FIG. 12a describes a process used in one embodiment to revoke access. A patient logs in to the patient portal using the logon process described above once they have a registered account based on email address (for example) 1201.

The user may then be presented with packages stored in the cloud that are associated with that patient's email address 1202. The patient then selects the images and/or reports, for example virtual packages containing DICOM studies, that are saved in the cloud 1202. The patient then may send a request to the cloud to delete the selected packages 1203. The cloud service then deletes the selections 1204 from the cloud database, or alternatively remove all access to the information 1204.

A similar process can be used in some embodiments to allow the patient to send the selected package or revoke access to the package. For example, the patient may wish to send his medical information to his doctor. The patient may instead type in his doctor's email address and hit send. The cloud service would then send an email to his doctor to access the patient's images (and in some embodiments create an account if the doctor has no account). Likewise, if a doctor or MDR already had access to a patient's medical information, the patient may remove access to the medical information by a specific entity. For example, the patient may select a package and be sent from the cloud service a list of all users with access to the package. The patient may then be able to select one or more users to remove from access to the particular virtual package.

### User Interface for Multiple Affiliations for Accounts

Unique identifiers are used to identify accounts within the cloud service. The preferred embodiment uses an email address as a unique identifier. An email address can be used for the login identifier for each user to login to the system. Other attributes could also be used, for example, requiring a unique username for the cloud service. Email address has a number of advantages for usability purposes. For example, using an email address as an account identifier and for logon purposes is advantageous because it does not change between websites/network services. The same email address can be used for the account name at multiple websites and is thus easy for a user to remember. Using specific usernames for a specific website lowers usability because the user must remember the specific username he used to sign up for the website. When he did sign up, his preferred username may have been taken, so the user may have to try a number of different account name/password combos to login to the cloud service, and may have to access “forgot your account name” functionality of the cloud service and reset the account. This concept especially applies to a website that hosts medical data because it may not be visited on a regular basis. Instead, many users may store their images within the website indefinitely and only revisit the site when access by the patient's medical servicers is required.

Each user (each with a corresponding unique identifier) may have multiple roles associated with that identifier. For example, suppose John Watson is a doctor at ABC hospital. He may review images and medical reports for multiple reasons, including as a physician for ABC hospital, as a radiologist on contract for County Medical Hospital, as a private consulting physician for third parties, as a doctor friend, and as a patient. Each of these roles allows a single account to review medical information sent to various organizations. For example, John Watson's account has access to images sent to ABC Hospital, to County Hospital, to his business as a consulting physician, to himself as normal “email” recipient, and to himself as a patient. Each of these roles may be considered an “inbox” by the cloud service system, and multiple accounts may have access to these inboxes. For example, other doctors at ABC Hospital may have access to the ABC Hospital cloud service inbox where they can view packages containing medical information that were sent to ABS Hospital.

FIG. 13 shows one embodiment of a user interface that allows for viewing inbox divisions. The figure demonstrates that cloud studies may be searched by a variety of criteria, including patient name, description, patient ID, DICOM accession number, among other search possibilities covered by the scope of the invention. After performing a search, the search results would be presented within the user interface 1302 listing a variety of information about each virtual package/DICOM study, including the number of days before deletion from the service, the name of the patient used in the DICOM study, the ID of the virtual package/study, the birth date of the patient, the patient's sex, the modalities used to gather the images or reports, the date of the package/study, a description of the study, and which inbox/organization the study was received for. The interface may allow a user to download the package in DICOM format by clicking on the package ID. As discussed above, downloading the package may automatically reconcile the data into the downloading hospital's system, as disclosed under the discussion for FIGS. 4 and 8.

The example interface shown in FIG. 13 also allows a user to click or touch the magnifying glass to view the medical information, including the images and/or reports within the package. These images may be transferred to a web browser or to a custom application installed on a workstation or mobile device affiliated with the user or organization that has access to the images within the cloud service. These applications may view and interact with DICOM compliant images and reports.

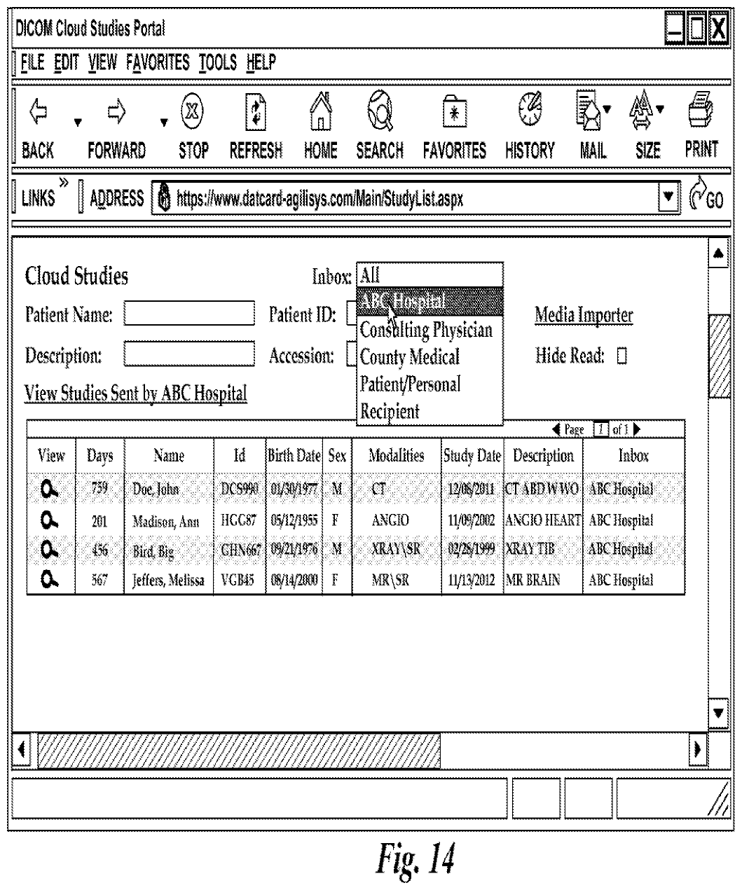

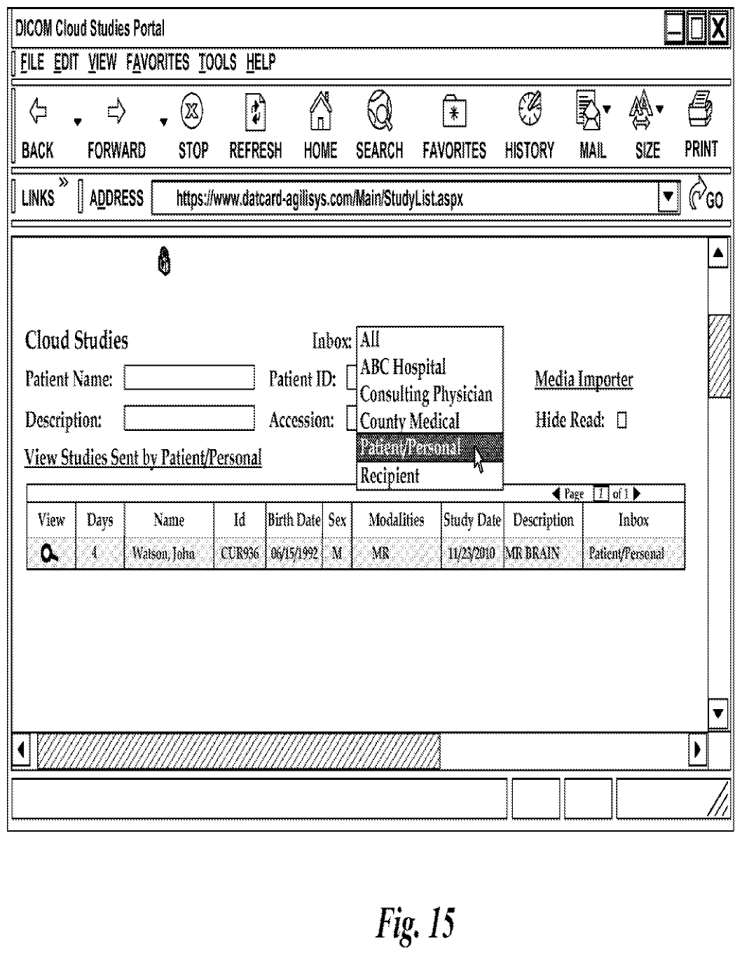

An example interface element such as the one depicted in 1303 allows a user of the cloud service to toggle between viewing the medical data received for all inboxes, or viewing the medical data received on a single inbox. For example, by default the cloud service may display all of the studies/packages that the user has access. The user may select to view only the studies within a specific inbox by selecting that inbox using a user interface element, such as the one shown in 1303. FIGS. 14 and 15 show how this embodiment displays only those studies sent to ABC Hospital (in FIG. 14) or John Watson's personal patient inbox (in FIG. 15) when those inboxes are specifically selected by the user interface element.

Media Importer may also be launched from this user interface. Media importer, made by DatCard Systems, Inc., is a utility that may assist a user in importing or exporting DICOM studies to and from the cloud, and to and from a PACS, including importing a DVD/CD ROM into an MDR's PACS. The documents about Media Importer, including “The Management of Outside CDs” and “DCS Media Importer User Manual” are hereby incorporated by reference and are also attached hereto made a part of this application.

### Tracking

In some embodiments, actions taken by users or recipients interacting with the cloud servers may be tracked and searched by administrators, users and recipients. These actions may be recorded within the cloud database, which may track what action was taken, by whom the action was taken, when the action was taken, about what the action was taken on, etc. For example, when a virtual package or one or more studies (containing images and/or reports) is uploaded to the cloud, the cloud server may record in the cloud database that an upload occurred, which user uploaded the studies, which studies where uploaded, when the upload occurred, if there was any intended recipient(s) for the upload, which computers were used to send the upload, etc. The same data may be recorded when a user accesses virtual packages or studies on the cloud server, views virtual packages or studies on the cloud server, downloads virtual packages or studies from the cloud server, prints images or reports within virtual packages or studies, etc. Other data that may be collected and tracked is a log of all activities taken in a single user session and/or the length of a user session with a cloud sever.

The tracking data that is stored in the cloud database may be searched by the administrator, or any user of an organization with the appropriate role or rights to search this audit information. Searching this information by, for example, various user, study, package, and time characteristics (such as by date, user, user/email address, package ID, study ID, organization, type of action taken, etc.) may return results that can be viewed by an administrator. This provides an advantage by allowing the dissemination and access of medical information to be tracked and audited, as potentially required by HIPAA regulations.

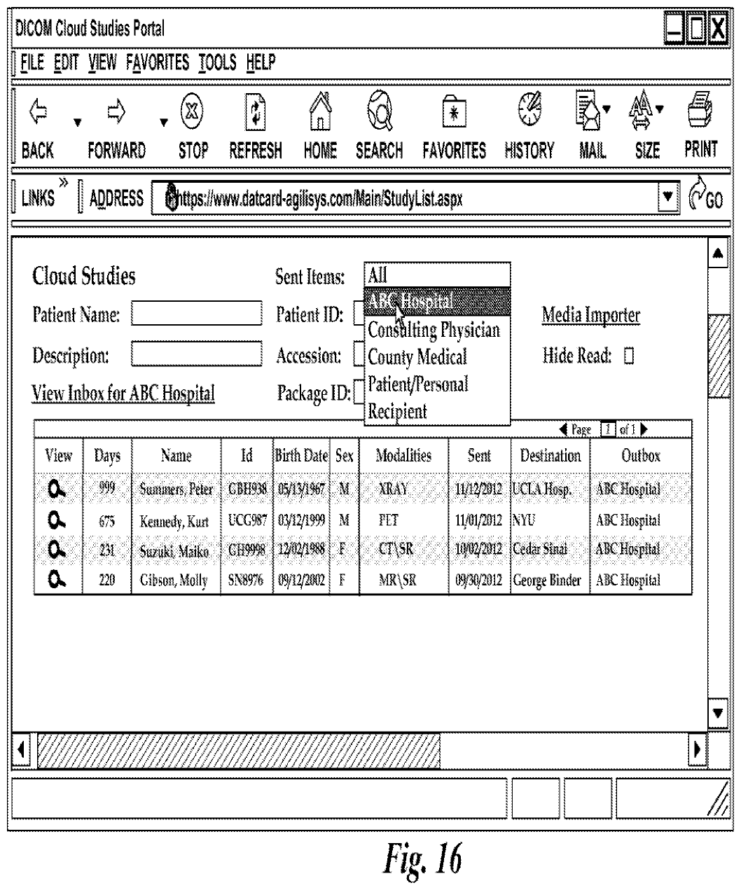

In some embodiments, tracking may be performed by viewing all packages sent by a specific organization or inbox. For example, in FIG. 14, a user may select “View Studies Sent by ABC Hospital” which may provide an example interface such as that depicted in FIG. 16, where all packages sent by ABC Hospital can be listed and searched within one interface. This interface has the same functionality and disclosure as FIG. 13, but also may include the date the package was sent by the organization or user and list the destination user/inbox that the package was sent to.

### Scanning

As known in the art, when medical images are sent between institutions, it is often advantageous to send along with the medical images with a diagnostic report based on an analysis of the images. Often times the report and the images are created by different parties or using different technical systems. It is not uncommon for the images to be transported as DICOM images, and the report to be faxed or sent via paper. Thus, it may be advantageous when importing image and reports into a PACS or the cloud to scan the report into a digital format and add it to the package or DICOM study it is associated with.

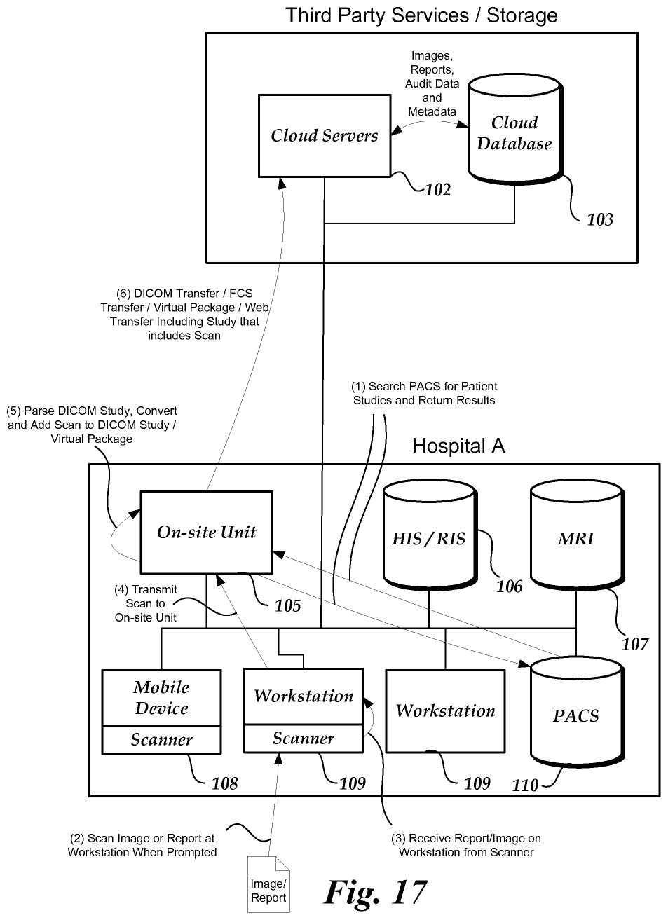

FIGS. 17 through 20 depict various sample embodiments for scanning a report (or alternatively any image or textual data) while uploading related medical data such as DICOM images to a cloud, creating portable media of the related medical data, or storing the related medical data onto a PACS or other device at an MDR. In all of these figures, a scanner is used to scan the medical images, report text, or other medical data into a DICOM image or DICOM structured report, depending on what time of data is being scanned.

The scanner involved may be attached to the workstation, as shown in FIG. 17. For example, it could be a USB or Bluetooth device that is connected and operates with workstation 109. The workstation processes any scanning done and operates the scanner. It also does any sending and receiving of the scan over the network. Alternatively, it could be a network attached device that has the ability to process scanned images and text and send and receive scans over the MDR's network. The scanner attached to Mobile Device 108 may be embedded. For example, a camera lens operated on a mobile phone with a sufficient quality for diagnosis if needed may operate as a scanner in some embodiments. Software on the workstation or the mobile phone may process the scans prior to transmitting them to the on-site unit, PACS or cloud servers depending on the embodiment.

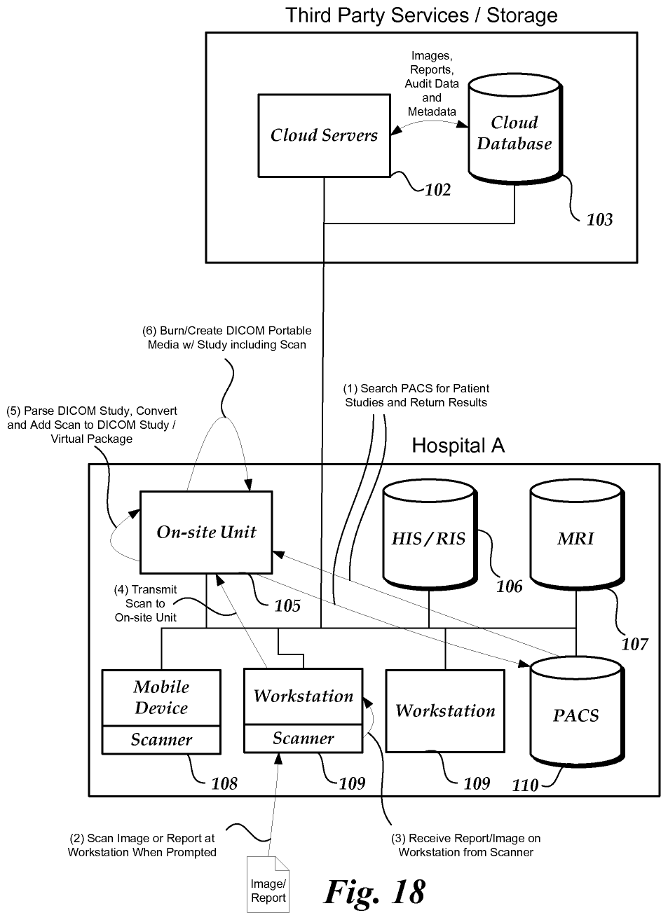

FIGS. 17 and 18 depict embodiments where a scan is performed of a related report prior to upload of DICOM images to the cloud or burning onto a CD. In step (1), the PACS (or any healthcare IT database) is searched for images based on criteria specified by a user or automated process. For example, a user at workstation 109 may be using the web interface on the on-site unit to search for DICOM studies that match a certain patient name, ID, or other criteria. The PACS is searched and returns a resulting image set. These images may then be retrieved by the on-site unit either now, or later, but in any case, prior to upload to the cloud. This may be no different than the process described in FIG. 3c, except that the system may additionally query the user to determine whether a scan should be added to a DICOM study in the package prior to uploading to the cloud or burning on a portable media.

In step (2), which may not need to be performed in this order, the report is scanned by the scanner attached to the workstation. That image is transferred from the scanner to the workstation (3). These images may then be transferred to the on-site unit from the workstation. Alternatively, the scanner, if stand alone, may transmit the images to the on-site unit itself.

In step (5), the DICOM study selected by the patient may be parsed for patient and MDR information, which is discussed further herein. The scanned data, if an image, is then converted to a DICOM image format and inserted into the selected DICOM image study. Alternatively, if the scanned data is a report, it may be converted to a DICOM structured report and inserted into the selected DICOM study.

The selected DICOM study may now be uploaded to the cloud service in step (6) (FIG. 17) or burned to a portable medium such as a CD, DVD, or USB key (FIG. 18) as described in FIG. 3c.

The order of operations depicted in the figures are not required for the invention to function. For example, all of the scanning may be done up front before the related DICOM study is searched for or selected.

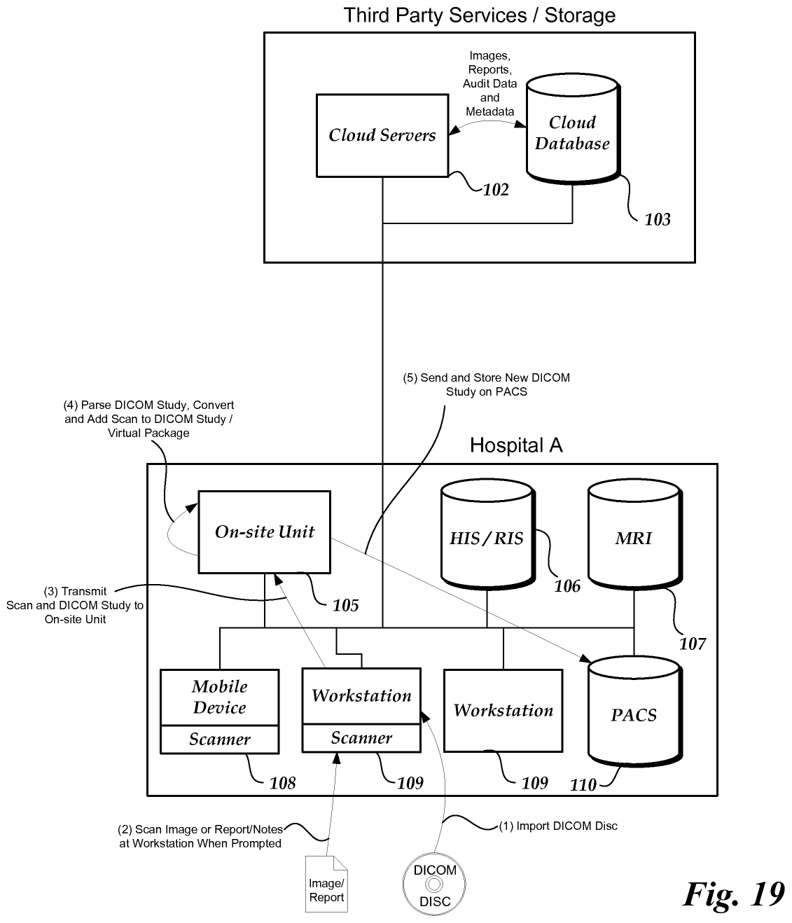

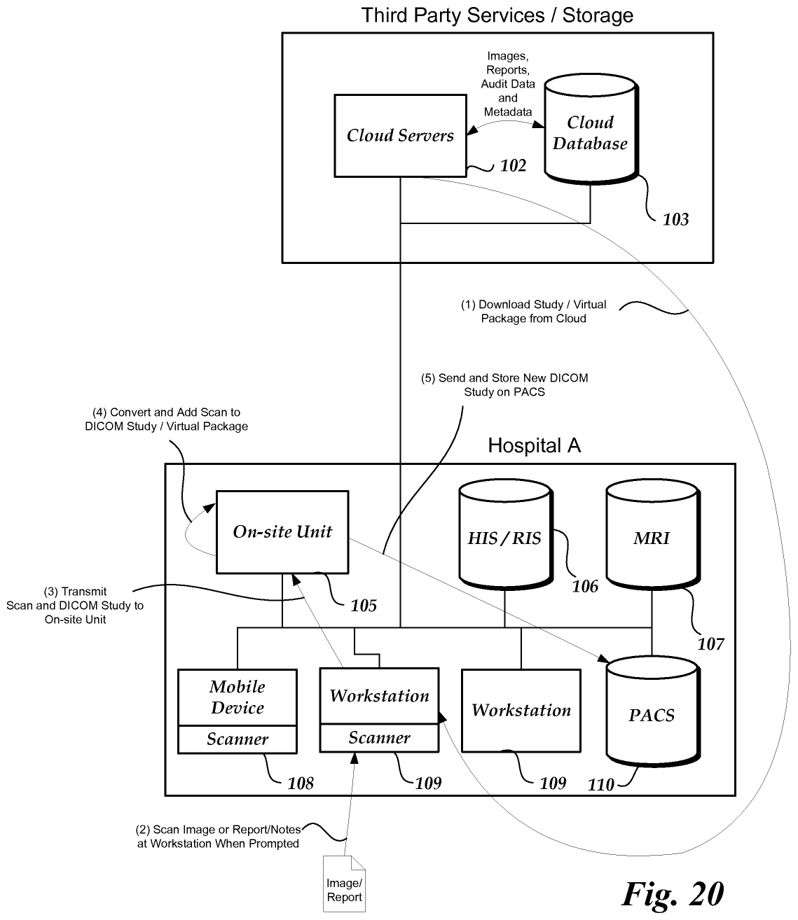

Other embodiments may use scanning to add to DICOM studies to be archived to an MDR's PACS or other medical data storage system. For example, FIGS. 19 and 20 describe one embodiment of a process and system that can be used to add scanned report(s) or image(s) to DICOM studies and store those modified studies on a portable medium or to a PACS. In the illustrative embodiment depicted in FIG. 19, a CD/DVD with DICOM images has been given to an MDR. Staff at the MDR begin the import process by inserting the media into a workstation. At this point, the workstation may communicate with the on-site unit and is prompted to select the files on the CD and begin importing the DICOM study. The DICOM data can be transferred at this point to the on-site unit, or transfer can occur later at step (3). In step (2), the image or report to be scanned is scanned by the workstation's scanner. In step (3), the combined DICOM images and the non-DICOM image or report are then transferred to the on-site unit for conversion to DICOM. Furthermore, the DICOM data may be translated for reconciliation into the MDR's PACS, as discussed under FIGS. 4 and 8. In other words, using techniques discussed herein, the patient name, patient ID, and other information contained in the DICOM study may be altered so that all information is compliant with the current MDR's patient records rather than using the patient IDs and MDR information of the sender.

In step (4), the DICOM study may be parsed for patient and MDR information, which is discussed further herein. The scanned data, if an image, is then converted to a DICOM image format and inserted into the selected DICOM image study. Alternatively, if the scanned data is a report, it may be converted to a DICOM structured report and inserted into the selected DICOM study. The entire DICOM study, with the newly added scanned information can then be transferred from the on-site unit to the PACS in step (5) for permanent storage. Alternatively, step (1) can be performed by transferring a DICOM study from the cloud service rather than using a CD. This DICOM study would then incorporate the newly scanned image or report once the process has completed and can be stored on the PACS.

As an alternative, the on-site unit need not be used. Instead, software local to the workstation may scan, parse, convert and insert into the DICOM study the newly scanned DICOM data. The workstation may then upload this data into the PACS or wherever it is being stored or archived in the MDR.

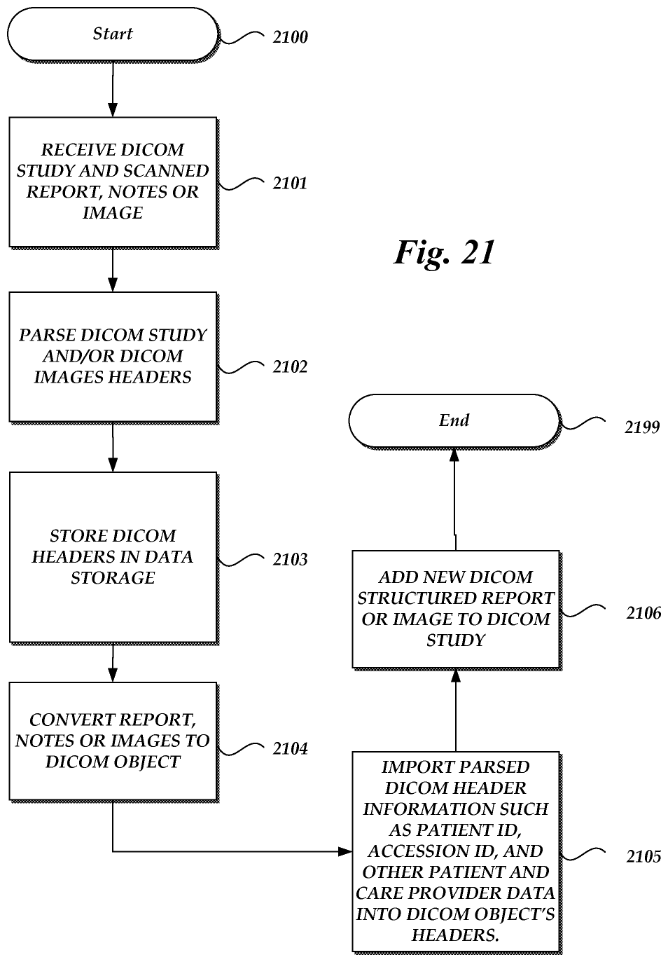

In order to determine what the DICOM header values should be for the newly scanned data, the on-site unit, workstation, or cloud system may scan the current DICOM study to be imported (pre or post reconciliation) in order to determine seed values for the newly scanned DICOM data. FIG. 21 depicts a sample embodiment that can read from a template DICOM study and use those values for a newly scanned DICOM image or structured report.

In block 2101, the parsing computer receives the template DICOM study and the scanned report or image. The DICOM study is then parsed and strips out DICOM headers based on the predetermined DICOM format 2102. Thus, values such as patient ID, accession ID, and patient name will be revealed by such parsing. These headers may then be stored in memory, cache, or more permanent storage 2103 local to the process performing the parsing. If not converted already, the new report or image can be converted to a DICOM compatible object, for example, a lossless JPEG image used for DICOM images, or a DICOM structured report. The headers of these DICOM objects are then populated with the parsed information from the DICOM study 2105. The new DICOM image or structured report is then added to the DICOM study, which involves minor alterations to the DICOM study object as known to those in the art. The DICOM study is then ready to be stored either in a CD/DVD or USB stick, PACS, or transferred to the cloud.

### Terminology

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The steps of a method, process, routine, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor such that the processor can read information from, and write information to, the storage medium. In the alternative, the storage medium can be integral to the processor. The processor and the storage medium can reside in an ASIC. The ASIC can reside in a user terminal or mobile device. In the alternative, the processor and the storage medium can reside as discrete components in a user terminal or mobile device.

Conditional language used herein, such as, among others, “can,” “could,” “might,” “may” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Conjunctive language such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments of the inventions described herein can be embodied within a form that does not provide all of the features and benefits set forth herein, as some features can be used or practiced separately from others. The scope of certain inventions disclosed herein is indicated by the appended claims rather than by the foregoing description. All changes which come within the meaning and range of equivalency of the claims are to be embraced within their scope.

What is claimed is:

1. A system for storing medical information remotely, the system comprising:
one or more processors;
a computer-readable memory;
a permanent electronic storage, wherein the permanent electronic storage is configured to store one or more DICOM objects;
a robotic burner; and
an export module comprising executable instructions stored in the computer-readable memory, wherein the one or more processors are programmed by the export module to at least:
receive, from a medical information database, one or more DICOM objects generated by a modality,
store the one or more DICOM objects on the permanent electronic storage;
receive, from a web browser operated by a user, an indication to perform one of: burning the DICOM objects to a portable media, or uploading the DICOM objects to a cloud service;
receive, from the web browser, an image or report related to the DICOM objects;
convert the image or report to a second DICOM object, and add the second DICOM object to the one or more DICOM objects;
compile a virtual package of the DICOM objects generated by the modality;
receive at least one recipient from the web browser that indicates the recipient account authorized to view the images within the cloud service, wherein the at least one recipients are identified by email address; and
transfer the one or more DICOM objects and the one or more recipients to the cloud service for storage, wherein the cloud service is configured to store the one or more DICOM objects for at least one day.

2. A method for sending medical information to a remote third party repository, comprising:
receiving DICOM patient data from a computing device within a medical data repository about a first patient;
receiving a recipient identifier from a user computing device identifying a third party able to access the DICOM patient data;
determining a HIPAA release that is required to be agreed to by the patient;
receiving an email address associated with the patient;
sending a URL link to an email address associated with the patient;
receiving a request for access of the URL link and a digital signature agreeing to the HIPAA release accessible by the URL link;
storing an identifier of the particular HIPAA release agreed to by the patient;
making the DICOM patient data available for download to the third party via a web interface;
searching for data associated with the third party that identifies the patient; if data associated with the third party that identifies the patient is found, translating the DICOM headers of the DICOM patient data to contain identifying information comprised of the data associated with the third party that identifies the patient.
